(19) United States
(12) Patent Application Publication
(10) Pub. No.: US 2002/0045219 A1
(43) Pub. Date: Apr. 18, 2002

(54) Production of human monoclonal antibodies

(75) Inventors: Scott K. Dessain, Brookline, MA (US); Richard A. Goldsby, Leverett, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Whitehead Institute for Biomedical Research, Cambridge, MA (US)

(21) Appl. No.: 09/759,984

(22) Filed: Jan. 12, 2001

Related U.S. Application Data
(60) Provisional application No. 60/222,473, filed on Aug. 2, 2000.

(51) Int. Cl. C12P 21/04; C12N 5/06

(52) U.S. Cl. 435/70.21; 435/328

(57) ABSTRACT

Hybridomas produced from human B-lymphocytes and other human (non-B lineage) cells and an ectopically expressed telomerase gene; mammalian cell lines that ectopically express telomerase and methods of using such cell lines in producing novel hybrid cells (hybridomas) that produce human monoclonal antibodies; human monoclonal antibodies produced by such novel hybridomas and DNA constructs useful for producing mammalian cell lines that ectopically express telomerase.

### RELATED APPLICATION(S)

This application claims the benefit of U.S. Provisional Application No. 60/222,473, entitled, “Production of Human Monoclonal Antibodies” by Scott K. Dessain and Richard A. Goldsby, filed on Aug. 2, 2000. The entire teachings of the referenced provisional application are incorporated herein by reference.

### GOVERNMENT SUPPORT

The invention was supported, in whole or in part, by grant number K08 HL04463-01 from National Institutes of Health. The Government has certain rights in the invention.

### BACKGROUND OF THE INVENTION

Antibodies are the effector molecules of the humoral immune response in mammals (1, 2). Also known as immunoglobulins (Ig), they are produced by B-lymphocytes in response to antigen stimulation. Each B-lymphocyte produces an antibody with a defined specificity for a particular antigen. During an infection, an individual will generally produce multiple unique B-lymphocyte clones, each expressing and secreting a single type of antibody directed at an antigen expressed by the infectious organism. Following the resolution of the infection, the newly-generated B-lymphocytes enter a quiescent state characterized by minimal proliferation and antibody secretion. These quiescent B-lymphocytes can last for the lifetime of the individual and serve as an immunological memory that can be quickly tapped should the individual again encounter the same infectious organism.

The development of monoclonal antibody technology in the 1970s greatly facilitated the study of antibody biology and the adaptation of antibodies for use in research and medicine (1, 3). Monoclonal antibodies are produced by hybrid cells that result from a fusion between normal B-lymphocytes and myeloma cells. The myeloma cell lines used for fusion are B-lymphocyte tumor cell lines that grow well in vitro and can propagate indefinitely, in contrast to normal B-lymphocytes that cannot replicate or produce antibody in vitro for more than a few days. Cells derived from a fusion of the two types of cells combine the in vitro growth characteristics of the myeloma cell line with the production of an antibody derived from the B-lymphocyte.

Hybrid cells (hybridomas) are generally produced from mass fusions between murine splenocytes, which are highly enriched for B-lymphocytes, and myeloma “fusion partner cells” (1, 2). The cells in the fusion are subsequently distributed into pools that can be analyzed for the production of antibodies with the desired specificity. Pools that test positive can be further subdivided until single cell clones are identified that produce antibodies of the desired specificity. Antibodies produced by such clones are called monoclonal antibodies.

Monoclonal antibodies have many advantages that make them particularly useful in research and medicine. They can be produced in large quantities and often have high and specific affinities for their particular antigens. However, their enormous potential utility is counter-balanced by the difficulty in producing antibodies suitable for pharmaceutical use. This is because the current state of the art for monoclonal antibody production is most effective in the production of murine antibodies. Murine antibodies are recognized by the human immune system as foreign. Patients may have allergic or anaphylactic reactions to the antibodies, or may develop their own antibodies directed against the murine antibodies. This can lead to the formation of large immune complexes that can precipitate in tissues and cause serum sickness, a syndrome consisting of fever, muscle and joint aches, rash, and renal and cerebrovascular injury. Consequently, murine antibodies are of limited value for use in humans.

Many investigators have attempted to generate human monoclonal antibodies by generating hybridomas with human B-lymphocytes (4, 5, 6, 7, 8, 9). Unfortunately, hybrid cells exhibit poor growth in vitro, low levels of antibody expression, instability of antibody expression, and a poor ability to be cloned by limiting dilution. The explanation for these phenotypes has not been elucidated. Accordingly, most investigators have concluded that the production of human monoclonal antibodies through the generation of hybrid cells formed with human B-lymphocytes is not feasible.

Consequently, diverse and cumbersome approaches have been used to produce human monoclonal antibodies. These include “humanizing” mouse antibodies by creating hybrid murine/hybrid immunoglobulin genes and generating antibodies in transgenic mice that bear human immunoglobulin gene loci. However, these methods are only able to produce antibodies that have been generated in mice by the murine immune system. They do not allow the isolation, production, and use of the naturally-occurring antibodies, the immunological memory that the human immune system produces in response to infections and other antigen exposures. The ability to make monoclonal antibodies directly from human B-lymphocytes is therefore needed and would be of considerable value.

### SUMMARY OF THE INVENTION

The present invention relates to a method of making human monoclonal antibodies through the use of novel hybrid cells. In the invention, hybrid cells are created by combining three elements: a fusion partner cell, a fusion cell (in particular a human B-lymphocyte), and an ectopic telomerase gene. As described herein the expression of an ectopic telomerase gene in hybrid cells formed from primary human B-lymphocytes and fusion partner cells improves their growth rate, level of Ig expression, stability of Ig expression, and the ability to be cloned by limiting dilution.

The present invention has made it possible to produce human antibody-secreting hybridomas directly from native human B-lymphocytes. Native human antibodies have isotypes and antigen specificities selected by the human immune response for their efficacy against pathogenic organisms and other foreign antigens. Indeed, many antibodies that are made by the human immune system in response to infections are potent enough to provide life-long immunity. Any person who has recovered from an infection is a reservoir of B-cells encoding antibodies that may be useful as therapeutics to protect another individual suffering from the same infection. For the first time, the present invention makes all of those antibodies accessible for use in research and medicine.

In addition, the present method for making human antibodies is much less cumbersome than previously available methods.

The present invention is based on a number of novel observations that explain the obstacles that have prevented others from efficiently generating human monoclonal antibody-secreting cells by cell fusion with primary human B-lymphocytes.

As described herein, it has now been shown that hybrid cells formed between cells that express the murine telomerase gene (mTERT) and primary human B-lymphocytes do not express the human telomerase gene (hTERT). This is surprising, particularly in light of the well-established ability of human B-lymphocytes to express hTERT when stimulated to proliferate in vivo and in vitro (10).

The telomerase enzyme protects the integrity of chromosomes by maintaining the length of telomeres, the complex DNA/protein structures that cap the ends of chromosomes. Telomerase can also inhibit apoptosis, the process of programmed cell death. The phenotypes of hybrid cells generated by fusion of primary human B-lymphocytes using presently available methods reflect a deficiency of hTERT. Applicants have shown that hTERT expression in such hybrid cells is sufficient to overcome the deficiencies seen with the previous hybridomas. Hybrid cells ectopically expressing hTERT have improved growth rates, levels of Ig expression, stability of Ig expression, and ability to be cloned by limiting dilution.

The present invention relates to hybrid cells created by combining three elements: a fusion partner cell, a fusion cell (in particular a human B-lymphocyte), and an ectopic human telomerase gene. It relates to methods of creating the novel hybrid cells, to the use of the novel hybrid cells to produce human antibodies, and to the human antibodies produced by such cells. The present invention makes it possible to produce human antibody-secreting hybridomas from native human B-lymphocytes.

In one embodiment of the present invention, an ectopic telomerase gene is introduced into the fusion partner cell line prior to fusion with human B-lymphocytes. The resulting hybrid cells express the ectopic telomerase gene. The present invention relates to mammalian cell lines that ectopically express telomerase and are useful in producing hybrid cells by fusing them with primary human cells; methods of using such cell lines in producing novel hybrid cells; methods of producing human antibodies through the use of the hybrid cells, human antibodies produced by the hybrid cells; and DNA constructs useful for producing mammalian cell lines that ectopically express telomerase.

In one embodiment of the present invention, the fusion partner cell line expresses an endogenous telomerase gene in addition to the ectopic human telomerase gene. Cell lines that ectopically express telomerase in addition to expressing an endogenous telomerase gene in the formation of hybrid cells have not been described previously. The present invention relates to mammalian cell lines that ectopically express telomerase in addition to an endogenous telomerase gene and are useful in producing hybrid cells by fusing them with primary human cells; methods of using such cell lines in producing novel hybrid cells; methods of producing human antibodies through the use of the hybrid cells, human antibodies produced by the hybrid cells; and DNA constructs useful for producing mammalian cell lines that ectopically express telomerase in addition to an endogenous telomerase gene.

Described herein is a method for creating or producing cell lines, referred to as fusion partners or as fusion partner cell lines, that ectopically express telomerase (e.g., by introducing an exogenous gene encoding telomerase, activating a silent endogenous gene or enhancing/prolonging expression of an endogenous gene that is normally expressed), and are useful to produce hybrid cells in which the ectopic telomerase gene is expressed.

Fusion partners can be produced by introducing an exogenous gene (a transgene) that encodes telomerase into a cell line in such a manner that the ectopic gene is expressed in hybrid cells formed by fusion of the fusion partners with fusion cells. For example, a telomerase-encoding gene can be introduced into a cell line under the control of a promoter that is not repressed in the hybrid cells (e.g. under the control of a retroviral promoter). In one embodiment, the fusion partner cell line is based on a murine myeloma cell line, into which a transgene encoding the human telomerase catalytic subunit gene (hTERT) was introduced. The hTERT transgene is under control of a promoter that maintains hTERT expression in hybrid cells produced by fusion of the fusion partner cell with a fusion cell. In one embodiment, the murine myeloma cell line is SP2/0, and the resulting fusion partner cell is referred to SP2/0 MP-hTERT.

The SP2/0 MP-hTERT cell line has been fused with normal human B-cells to produce hybrid cells (hybridomas) that express human antibody molecules and express an ectopic hTERT. Introduction of an ectopic hTERT into the fusion partner cell line has proved to be an efficient means of inducing ectopic hTERT expression in the hybrid cells, obviating the need to introduce an ectopic hTERT into the hybrid cells during or after their creation. The SP2/0 MP-hTERT cell line forms productive fusions with human B-lymphocytes at high frequency. The resulting hybrid cells are superior to hybrid cells made using presently available techniques with regards to growth rate, level of Ig expression, stability of Ig expression, and the ability to be cloned by limiting dilution.

The SP2/0 MP-hTERT cell line has also been fused with normal murine B-cells to produce hybrid cells (hybridomas) that express murine antibody molecules and express an ectopic hTERT. Applicants have found that the ectopic expression of hTERT in the SP2/0 cells prior to their fusion with primary cells improved their ability to form hybrid cells, relative to the presently available methods, as reflected by an increase in the number and sizes of hybrid cell colonies obtained. This may reflect the observation that ectopic hTERT expression reduces the tendency of SP2/0 cells to die spontaneously under high-density culture conditions (Example 7). The increased efficiency in establishing clones and the overall increase in clone size engendered by ectopic hTERT expression are superior to those of the presently available methods of creating murine hybridomas. Therefore, the present invention is also an improvement in the production of murine monoclonal antibodies.

In another embodiment, the fusion partner is a human cell line expressing an ectopic hTERT, or an endogenous hTERT that is regulated in such a manner that it is ectopically expressed in hybrid cells formed by fusion of the fusion partner cell and a primary fusion cell. Many existing HAT-sensitive human fusion cell lines, and additional lines that can easily be made using standard techniques, can be modified to express an ectopic hTERT. Antibodies produced by hybrid cells formed between human fusion partners and human B-lymphocytes will bear only human patterns of glycosylation (11). Such antibodies may be less immunogenic and more stable in vivo than antibodies made in murine or murine/human hybrid cells.

The antibodies produced by hybridomas of the present invention are native human antibodies and, thus, presumably have already been optimized in vivo for effective cooperation with other components of the human adaptive and innate immune systems in the response to infection. The present invention makes it possible to harvest the full panoply of human antibody diversity, representing all antibody isotypes, allotypes, and virtually infinite antigen specificities that comprise the human antibody repertoire. It will enable the isolation, study, production, and use of the antibodies that comprise the B-cell immunological memory in humans. It will dramatically expand the utility of antibodies in biology, chemistry, biotechnology, pharmacy, and medicine.

The novel hybrid cells described herein maintained the growth characteristics of the myeloma cell line but produced antibody molecules derived from the genome of the normal B-lymphocyte. In essence, the hybrid cells combined in vitro growth characteristics of the cell line with phenotypic characteristics derived from the fusion cell. This observation gives rise to a model for the efficient generation of a variety of hybrid cells. Fusion partner cell lines of any cell lineage that express an ectopic telomerase gene should make it possible to produce stable hybrids through fusion with fusion cells, including primary cells, derived from many sources.

Thus, the following invention relates to a method of producing a hybrid cell that expresses an ectopic telomerase gene comprising fusing a fusion partner cell with a fusion cell under conditions appropriate for the production of a hybrid cell, wherein the ectopic telomerase gene is introduced into one of the cells and is expressed in the hybrid cell, thereby producing a hybrid cell that expresses an ectopic telomerase gene. In one embodiment of the method, the ectopic telomerase gene is introduced into the fusion partner cell prior to fusing the fusion partner cell with a fusion cell. In a further embodiment, the ectopic telomerase gene is introduced into the fusion cell prior to fusing the fusion cell with a fusion partner cell. In an additional embodiment, the ectopic telomerase gene is introduced into the fusion partner cell, fusion cell, or hybrid cell during the cell fusion. In an additional embodiment, the ectopic telomerase gene is introduced into the hybrid cell. The fusion partner cell, in one embodiment, is an immortal mammalian cell, such as a human cell or a murine cell. In one embodiment of the method, the fusion partner cell is a B-lineage cell or a myeloma cell line. In an embodiment, the fusion cell is a B-lineage cell, such as a B-lineage cell of human origin. As described herein, a wide variety of telomerase genes can be used and in a particular embodiment, the telomerase gene is the human telomerase gene. A hybrid cell produced by the method described, such as a hybrid cell that ectopically expresses telomerase, such as human telomerase, is also a subject of the present invention. Such hybrid cells (hybridomas) produce antibody molecules, such as human antibody molecules. The antibody molecules produced include, but are not limited to, immunoglobulin A, immunoglobulin E, immunoglobulin G, immunoglobulin M, or portions or derivatives thereof. Antibody molecule produced by hybrid cells of one embodiment of the present invention are encoded in full or in part by genes originating from the human B-cell fusion cell used to produce the hybrid cells.

A particular embodiment of the invention is a method of producing a hybrid cell by fusing: (a) a mammalian fusion partner that ectopically expresses telomerase and (b) a human B-lymphocyte, wherein the hybrid cell produced expresses an antibody derived from the human B-lymphocyte. Antibodies produced by the resulting hybrid cell are also a subject of this invention.

An additional embodiment of this invention is a method of producing a hybrid cell that expresses an ectopic telomerase gene comprising fusing an immortal mammalian cell line that expresses an ectopic telomerase gene with a fusion cell under conditions appropriate for the production of hybrid cells, thereby producing a hybrid cell that expresses an ectopic telomerase gene. The fusion partner cell is, in particular embodiments, an immortal mammalian cell, a human cell or a murine cell, such as a B-lineage cell or a myeloma cell line. The fusion cell is, for example, a B-lineage cell, such as a B-lineage cell of human origin. A variety of telomerase genes can be used in the method and in a particular embodiment, the telomerase gene is the human telomerase gene.

Hybrid cells produced by this method and particularly hybrid cells that ectopically express human telomerase, are also the subject of this invention. Hybrid cells produced by the method produce antibody molecules, particularly human antibodies. The antibody molecules produced include, but are not limited to, immunoglobulin A, immunoglobulin E, immunoglobulin G, immunoglobulin M, or portions and derivatives thereof. Hybrid cells produced by the method described produce, in one embodiment, antibody molecules encoded in full or in part by genes originating from the human B-cell fusion cell.

A further embodiment of the present invention is immortal mammalian cell lines that express an endogenous telomerase gene and an ectopic telomerase gene. The cell line can be, for example, of human or murine origin. In one embodiment, the immortal mammalian cell line has been modified to ectopically express telomerase constitutively by expressing a telomerase gene that is expressed from a constitutively active promoter. Immortal mammalian cell lines of the present invention can be B-lineage cell lines (e.g., of human or murine origin) that express an endogenous telomerase gene and an ectopic telomerase gene. In the embodiments in which the telomerase gene is expressed from a constitutively active promoter, the constitutively active promoter can be, for example, a viral promoter, a eukaryotic promoter, a prokaryotic promoter or a synthetic promoter. In one embodiment, the immortal mammalian cell line of B cell lineage express Epstein-Barr Virus antigens.

An additional embodiment of the present invention relates to an immortal mammalian B-cell (e.g., murine or human), for production of hybridomas, wherein telomerase is expressed ectopically. The immortal mammalian B cell, in one embodiment, has been modified to ectopically express telomerase constitutively by expressing a telomerase gene that is expressed from a constitutively active promoter. An immortal mammalian lymphoblastoid cell that ectopically expresses telomerase is a subject of this invention.

Another embodiment of this invention is a method of producing a hybridoma that ectopically expresses telomerase, comprising fusing an immortal mammalian cell line that ectopically expresses telomerase with a fusion partner, under conditions appropriate for production of hybridomas, thereby producing a hybridoma that ectopically expresses telomerase.

In specific embodiments, the immortal mammalian cell line is a human cell line or a murine cell line and the fusion cell is a human cell. The immortal mammalian cell line can be, for example, a myeloma cell line (e.g., a murine cell line) and the fusion partner can be, for example, a B-lineage cell. The B-lineage cell can be, for example, an antigen-stimulated human peripheral blood mononuclear cell. Hybridomas produced by or resulting from the above described methods are also the subject of this invention.

An additional aspect of this invention relates to a method of producing human monoclonal antibodies, comprising maintaining a hybidoma that ectopically expresses telomerase under conditions appropriate for the production of monoclonal antibodies by the hybridoma, thereby producing monoclonal antibodies. In one embodiment, the invention relates to a method of producing human monoclonal antibodies, comprising: (a) fusing an immortal mammalian cell line that ectopically expresses telomerase with a human fusion partner, under conditions appropriate for hybridoma formation, thereby producing hybridomas that ectopically express telomerase and (b) maintaining hybridomas produced in (a) under conditions appropriate for production of monoclonal antibodies by the hybridomas, whereby human monoclonal antibodies are produced. In a particular embodiment, the immortal mammalian cell line (e.g., a human or murine cell line) has been modified to ectopically express telomerase constitutively by expressing a telomerase gene that is expressed from a constitutively active promoter. Hybridomas that ectopically express telomerase, such as murine hybridomas, human hybridomas and murine/human hybridomas, are also the subject of this invention.

A further subject of this invention is a method of producing a hybridoma that produces antibodies that bind an antigen expressed by a malignant cell, comprising fusing an immortal cell line that ectopically expresses telomerase with a fusion cell that is a malignant cell (e.g., a solid malignant tumor cell or a hematopoietic tumor cell) under conditions appropriate for formation of hybridomas, whereby a hybridoma that produces antibodies that bind the antigen expressed by the malignant cell is produced. The solid tumor can be any type of solid tumor, such as a gastrointestinal tumor, a breast tumor, a kidney tumor, a brain tumor, a liver tumor, a stomach tumor, a lung tumor, a pancreatic tumor, a tumor of the reproductive systems, a prostate tumor, an eye tumor, a skin tumor, a melanoma, adenomas, polyps, dysplasias, in situ carcinomas, or intra-epithelial neoplasms and the hematopoietic tumor cell can be, for example, leukemia, lymphoma, or myeloma, or myelodysplastic syndromes.

A method of producing a hybridoma that expresses antibodies that bind an antigen expressed by a pathogen is also a subject of this invention. The method comprises fusing an immortal cell line that ectopically expresses telomerase with fusion cells that are B-lineage cells from an individual who is or has been infected with the pathogen, under conditions appropriate for formation of hybridomas, whereby a hybridoma that produces antibodies that bind the antigen expressed by the pathogen is produced.

The pathogen can be any pathogen against which antibody production is desired, such as a RNA virus, a DNA virus, a bacterium, an intracellular parasite, a fungus, a helminth and a protozoan. The RNA virus can be, for example, a member of a RNA virus family selected from the group consisting of: Picomaviridae, Calciviridae, Togaviridae, Flaviviridae, Coronaviridae, Rhabdoviridae, Filoviridae, Paramyxoviridae, Orthomyxoviridae, Bunyaviridae, Arenaviridae, Reoviridae and Retroviridae. The DNA virus can be, for example, a member of a DNA virus family selected from the group consisting of: Hepadnaviridae, Parvoviridae, Papovaviridae, Adenoviridae, Herpesviridae and Poxviridae and Hepatitis. The bacterium can be, for example, selected from the group consisting of: gram-positive cocci, gram positive bacilli, gram-negative bacteria, anaerobic bacteria, organisms of the families Actinomycetaceae, Bacillaceae, Bartonellaceae, Bordetellae, Captophagaceae, Corynebacteriaceae, Enterobacteriaceae, Legionellaceae, Micrococcaceae, Mycobacteriaceae, Nocardiaceae, Pasteurellaceae, Pseudomonadaceae, Spirochaetaceae, Vibrionaceae and organisms of the genera Acinetobacter, Brucella, Campylobacter, Erysipelothrix, Ewingella, Francisella, Gardnerella, Helicobacter, Levinea, Listeria, Streptobacillus and Tropheryma. The intracellular parasite can be, for example, Chlamydiaceae, Mycoplasmataceae, Acholeplasmataceae, Rickettsiae or organisms of the genera Coxiella and Ehrlichia. The fungus can be, for example, Aspergillus, Blastomyces, Candida, Coccidioides, Cryptococcus, Histoplasma, Paracoccicioides, Sporothrix, organisms of the order Mucorales, organisms inducing choromycosis and mycetoma and organisms of the genera Trichophyton, Microsporum, Epidermophyton, or Malassezia. The helminth is selected from the group consisting of: Nematodes, Trematodes and Cestodes. The protozoan can be, for example, an organism of the phylum Sarocomastigophora, the phylum Apicomplexa, the phylum Ciliophora, the phylum Microspora, or Pneumocystis carinii.

This invention also relates to a method of producing a hybridoma that produces antibodies that bind a self-antigen, comprising fusing an immortal cell line that ectopically expresses telomerase with a fusion cell obtained from a person who is or has been affected by an autoimmune disease, under conditions appropriate for formation of hybridomas, whereby a hybridoma that produces antibodies that bind the self-antigen is produced. Such antibodies are useful, for example for treating auto-immune conditions.

This invention further relates to a method of producing a hybridoma that produces antibodies that bind a prion antigen, comprising fusing an immortal cell line that ectopically expresses telomerase with a fusion cell obtained from a person who is or has been affected by a prion disease, under conditions appropriate for formation of hybridomas, whereby a hybridoma that produces antibodies that bind the prion antigen is produced.

Also the subject of this invention is a method of producing a hybridoma that produces antibodies that bind an antigen in an antigen preparation, comprising fusing an immortal cell line that ectopically expresses telomerase with a fusion cell that has been stimulated in vitro in the presence of the antigen preparation, under conditions appropriate for formation of hybridomas, whereby a hybridoma that produces antibodies that bind an antigen in the antigen preparation is produced.

DNA constructs useful for introducing DNA that encodes telomerase into a mammalian cell to modify the cell to ectopically express telomerase are also the subject of this invention. Such DNA constructs comprise, for example, (a) a telomerase gene; and (b) DNA that undergoes homologous recombination with a region of genomic DNA of the mammalian cell in such a manner that introduction of the telomerase gene into the genomic DNA of the mammalian cell places it under control of transcription regulatory elements of the mammalian cell that direct constitutive expression of the telomerase gene in the mammalian cell. The DNA construct can be a plasmid or a viral vector. Further subjects of this invention are DNA constructs useful for introducing DNA to modify a mammalian cell to ectopically express an endogenous telomerase gene, comprising, for example, a constitutively active promoter flanked by DNA that undergoes homologous recombination with the genomic DNA of the mammalian cell in such a manner that the constitutively active promoter is introduced into a site from which it directs the constitutive ectopic expression of an endogenous telomerase gene in the mammalian cell. In one embodiment of the DNA construct, the DNA that undergoes homologous recombination with genomic DNA is homologous to DNA of the endogenous telomerase gene promoter. The construct can be, for example, a plasmid or a viral vector.

### BRIEF DESCRIPTION OF THE DRAWINGS

FIG. 1 shows an assessment of expression of hTERT and mTERT in hybrid cells formed between SP2/0 cells or SP2/0 MP-hTERT cells and pokeweed mitogen-stimulated peripheral blood mononuclear cells. RT-PCR was used to detect the presence of hTERT and mTERT mRNA in the hybrid cells. Upper lanes: mTERT expression. Middle lanes: hTERT expression. Lower lanes: GAPDH expression. Lane H: HS Sultan; lane S SP2/0; lane T: SP2/0 MP-hTERT; lane W: water-only control; lanes 1-4: hybrid cell populations SP 4.3, SP 4.4, MPT 73.1, MPT 6.2. SP 4.3 and 4.4 were formed with SP2/0 cells. MPT 73.1 and MPT 6.2 were formed with hTERT-expressing SP2/0 MP-hTERT cells. SP 4.3 and MPT 73.1 do not express human Ig; SP 4.4 and MPT 6.2 express human Ig (data not shown).

FIG. 2 shows an assessment of expression of telomerase catalytic activity in hybrid cells formed between SP2/0 cells or SP2/0 MP-hTERT cells and pokeweed mitogen-stimulated perpheral blood mononuclear cells. The TRAP assay was used to detect telomerase catalytic activity in the hybrid cells. Lane 1: Hela cell line positive control; lanes 2-5 hybrid cell populations SP 4.3, SP 4.4, MPT 73.1, MPT 6.2, as in FIG. 1. H: heat treated negative control for PCR contamination.

FIG. 3 shows an assessment of expression of an ectopic hTERT in SP2/0 cells. A Reverse Transcriptase-PCR (RT-PCR) assay to detect the presence of hTERT mRNA. Upper lanes: mTERT expression. Middle lanes: hTERT expression. Lower lanes: GAPDH expression. Lanes 1, 4: HS Sultan, an hTERT-expressing lymphoblastoid cell line; lanes 2, 5: SP2/0 murine myeloma cell line; lanes 3,6: SP2/0 MP-hTERT murine myeloma cells expressing an ectopic hTERT. Lanes 1-3: cDNA synthesis performed with Reverse Transcriptase. Lanes 4-6: mock eDNA synthesis performed in the absence of Reverse Transcriptase.

FIG. 4 shows a high rate of expression of hTERT in hybrid cells formed between SP2/0 MP-hTERT cells and pokeweed mitogen-stimulated perpheral blood mononuclear cells. RT-PCR was used to detect the presence of hTERT and mTERT MRNA in the hybrid cells. Upper lanes: mTERT expression. Middle lanes: hTERT expression. Lower lanes: GAPDH expression. Lane H: HS Sultan; lane S SP2/0; lane T: SP2/0 MP-hTERT; lane W: water-only control; lanes 1-8 hybrid cell populations 3.3, 3.4. 4.1, 4.2, 4.3, 4.4, 5.1, 5.2. All of the hybrid cell populations except 5.2 expressed human Ig (data not shown).

FIG. 5 show a graphic representation of the effect of ectopic hTERT expression on the survival of SP2/0 cells in vitro at high culture density. Murine myeloma cells SP2/0 (no ectopic hTERT) and SP2/0 MP-hTERT (with ectopic hTERT were seeded at confluent density; cells were evaluated daily for their ability to exclude Trypan Blue dye, an indicator of cell viability.

FIG. 6 is a schematic representation of the plasmid pMSCV Puro hTERT

### DETAILED DESCRIPTION OF THE INVENTION

1. Overview of the Invention

The present invention relates to a method of creating antibody-secreting hybrid cells from three elements: a fusion partner cell, a primary B-lymphocyte, and an ectopically expressed TERT gene. It further relates to antibody-secreting hybrid cells in which telomerase is ectopically expressed; antibodies produced by the hybrid cells; DNA constructs useful for producing hybrid cells of the present invention and methods of making and using each of the foregoing.

In a specific embodiment, the present invention relates to a method of creating antibody-secreting hybrid cells from three elements: a mammalian cell line, a primary B-lymphocyte, and an ectopically expressed TERT gene, in which an ectopic TERT gene is expressed in the mammalian cell line prior to fusion of the cell line with primary B-lymphocytes.

The present invention relates to mammalian cell lines that ectopically express telomerase and their use as fusion partner cell lines to produce hybrid cells (hybridomas) in which telomerase is ectopically expressed in such a manner that its transcription is not repressed in the hybrid cells; hybrid cells in which telomerase is ectopically expressed; antibodies produced by the hybrid cells; DNA constructs useful for producing mammalian cell lines of the present invention and methods of making and using each of the foregoing. The term “a mammalian cell line that ectopically expresses telomerase” includes cell lines/cells that have themselves been modified to ectopically express telomerase, as well as progeny and derivatives thereof.

The present invention relates to mammalian cell lines that ectopically express telomerase in addition to an endogenous telomerase gene and their use fusion as partner cell lines to produce hybrid cells (hybridomas) in which telomerase is ectopically expressed in such a manner that its transcription is not repressed in the hybrid cells; hybrid cells in which telomerase is ectopically expressed; antibodies produced by the hybrid cells; DNA constructs useful for producing mammalian cell lines of the present invention and methods of making and using each of the foregoing. The term “a mammalian cell line that ectopically expresses telomerase in addition to an endogenous telomerase gene” includes cell lines/cells that have themselves been modified to ectopically express telomerase, as well as progeny and derivatives thereof.

2. Scientific Basis for the Present Invention

As described below and in the Examples, Applicants have efficiently generated human monoclonal-antibody-secreting hybrid cells by cell fusion with primary human B-lymphocytes.

a. As described in Example 1, Applicants have demonstrated that hybrid cells formed between mTERT-expressing murine cells and primary human B-lymphocytes do not express the hTERT. This result is novel in at least two respects. First, hTERT expression in hybrid cells formed between TERT-expressing cells and primary B-lymphocytes, and in particular, primary human B-lymphocytes, has not been reported. Second, hTERT expression in hybrid cells formed between mTERT-expressing murine cells and primary human B-lymphocytes has not been reported.

b. As described in Examples 4-6, hybrid cells formed between an mTERT-expressing murine myeloma cell line and primary human B-lymphocytes appear to benefit from hTERT expression, manifested by improved growth rates, increased levels of Ig expression, improved stability of Ig expression, and an improved ability to be cloned by limiting dilution. These results imply that the difficulties others have encountered in attempting to generate hybrid cells that secrete human monoclonal antibodies were due to an inability of the hybrid cells to express hTERT.

This is surprising because, prior to Applicants' observations, there appeared to be no significant phenotypic differences between the mTERT and hTERT proteins (12). Both elongate telomeric substrates by synthesizing repeats of an identical DNA sequence, both contribute to the maintenance of telomere lengths in vivo, and both appear to be important contributors to the ability of some cells to propagate (13, 14). Nonetheless, as described above, the hybrid cells of the present invention benefit from hTERT expression in addition to mTERT expression. Despite their substantial similarities, mTERT and hTERT are not equivalent in their abilities.

c. As described in Examples 2-4, expressing an ectopic hTERT in a fusion partner cell line prior to its fusion with primary B-lymphocytes is an effective method for creating hybrid cells that secrete human antibodies and express an ectopic hTERT. No published studies have addressed the ectopic expression of hTERT in a cell in order to cause hTERT expression in hybrid cells derived from fusing that cell with other cells. Similarly, no published studies have addressed the ectopic expression of hTERT in a cell that already expresses an endogenous telomerase gene in order to cause ectopic hTERT expression in hybrid cells derived from fusing that cell with other cells. The use of such cells in cell fusions with primary human B-lineage cells in order to create hybrid cells that express an ectopic hTERT and secrete human antibodies has not been reported. Indeed, the use and utility of ectopic expression of any TERT gene in any cell to improve its ability to form TERT-expressing hybrids with any other cell have not been previously described.

d. As described in Examples 7 and 8, expression of hTERT provides unexpected phenotypic benefits to an mTERT-expressing murine cell line, the SP2/0 myeloma cell line. SP2/0 cells expressing hTERT are resistant to the spontaneous cell death that unmodified SP2/0 cells undergo when maintained at high-density culture conditions. In addition, SP2/0 cells expressing hTERT are more effective in forming hybrids with murine B-cells than cells that do not express hTERT, in that the hybrids form more frequently and tend to be of larger size. These results provide further evidence that mTERT and hTERT proteins are not functionally equivalent. Furthermore, they suggest that hTERT may generally improve the effectiveness of hybrid cell formation resulting from intra-species and inter-species fusions between fusion partner cell lines and fusion cells of murine or other non-human origin.

3. Definitions

The following terms, as used herein, have the following definitions, respectively: The word “antigen” refers to any agent of any form that can induce a B-cell-lineage response and the development of an immunoglobulin with specific binding affinity for the antigen.

The terms “B-cell” and “B-lymphocyte” are used synonymously to refer to B-lineage cells.

The phrase “cell fusion” and the term “fusion” refer to a mixture of fusion partner cells with fusion cells under conditions designed to facilitate the formation of fused cells (hybrid cells).

A “cell line” is a clonal or polyclonal population of cells that will propagate in vitro under appropriate culture conditions.

A “cloned cell line”, or “monoclonal cell line”, is a cell population in which every cell is an unmodified progeny of a single ancestor cell. A “monoclonal antibody” is an antibody produced by a monoclonal cell line.

The phrase “derived from” is used to denote the origin of the subject under consideration. An antibody that is “derived from human” is an antibody that is encoded at least in part by a human immunoglobulin gene. The term is used synonymously with the terms, “human antibody” and “human immunoglobulin”. A “fully human” antibody or immunoglobulin is one that has been produced entirely by genes and cells of human origin. A “fully human” hybrid cell is a cell formed between two human cells, although it may contain elements that are not derived from human but do not affect the nature of the produced antibody, e.g. an ectopic retroviral promoter or a non-human primate TERT gene. “Derived from” a particular source is also intended to include entities having substantially the same composition as entities originating from that source. For instance, a synthetic telomerase gene would be considered derived from human if it had a sequence similar to a native human telomerase gene. For example, a telomerase gene is derived from an organism or animal if it has been obtained or isolated from that organism or has the same or substantially the same sequence as the endogenous telomerase gene of the organism and has been made or synthesized (e.g., by recombinant DNA methods, synthetic methods).

The phrases “ectopic expression”, “ectopically expressed”, and “expressed ectopically” refer to gene expression that is under the control of transcription regulatory elements other than those that normally control the endogenous gene at its wild-type chromosomal site. Also included in the term is expression from an endogenous gene in cases in which a cell has been modified in such a way as to cause or enhance its expression in hybrid cells, including causing the expression of an endogenous gene that would otherwise be transcriptionally silent. This may include altering genetic or epigenetic control mechanisms in the cell in such a way as to subvert the normal regulation of the gene by its endogenous control sequences. One example of this would be to express a protein that would act on the endogenous regulatory sequences of a gene in order to induce expression of the gene.

An “ectopic gene” is a gene that is expressed ectopically. Such genes include exogenous genes that have been introduced into cells, in which they are expressed, and endogenous genes whose expression has been altered in such a manner that it differs from normal expression of the gene in the cell (e.g., its expression has been enhanced, prolonged or increased). “Ectopic expression of telomerase” is synonymous with “expression of an ectopic telomerase gene” and refers to the expression of telomerase protein from an ectopic telomerase gene.

An “endogenous gene” is a gene that exists within the genome of a cell as the cell is obtained.

A “flanked” DNA sequence is one that has homologous sequences on either or both sides of the DNA to be integrated into the genomic DNA. The homologous sequences can be immediately adjacent to the DNA to be integrated or separated by DNA sequences that are not homologous provided that they will not prevent homologous recombination from occurring.

The phrase “fusion cell” refers to a cell to be fused with a fusion partner in order to produce a hybrid cell.

The phrases “fusion partner” and “fusion partner cell line” refer to a cell line to be fused with other cells in order to produce hybrid cells. A “fusion partner cell” is a cell of the cell line intended to be fused with another cell in order to produce a hybrid cell.

The phrase “hybrid cell” is used synonymously with “hybridoma”, “hybrid”, and “fused cell” and refers to a viable cell that is the product of a fusion between a fusion partner cell and fusion cell.

The word “immortal” refers to the ability of a cell or a cell line to be propagated indefinitely when maintained in appropriate culture conditions.

The word “immunoglobulin” is synonymous with the word “antibody”. It is frequently abbreviated to “Ig”. “IgA”, “IgE”, “IgG”, “IgM” refer to different classes of immunoglobulin molecules.

The phrase “lymphoblastoid cell line” is a B-cell line that either has been infected with the Epstein Barr Virus (EBV) or expresses EBV antigens.

As used herein, a “primary cell” is a cell as removed from an organism without genetic or epigenetic modification. B-cells that have been removed from an organism and treated only with growth promoting agents are still considered to be “primary”.

The phrase “retroviral gene transfer” refers to the use of RNA viruses to introduce genes into a recipient cell. “Telomerase” is a protein produced by a telomerase gene. TERT refers to any telomerase-encoding gene; mTERT and hTERT refer to DNA encoding murine and human telomerase respectively. DNA can be genomic DNA, cDNA or synthetic or recombinantly-produced DNA.

4. Hybrid Cells that Express an Ectopic Telomerase Gene

The present invention relates to hybrid cells created by combining three elements: a fusion partner cell, a primary cell, and an ectopically expressed telomerase gene. Hybrid cells created by combining a cell of a mammalian cell line, a primary human B-lymphocyte, and an ectopically expressed human telomerase gene are useful as means of producing human antibodies.

The present invention is based in part on the observation that hTERT is not expressed in human/murine hybrid cells. Although human/murine hybrid cells express mTERT, they suffer from inadequate hTERT expression and benefit from the expression of an ectopic hTERT.

The phenotypes of murine/human and human/human hybrid cells created by the presently available methods are very similar (4, 5, 6, 7, 8, 9). Therefore, it is likely that that hybrid cells formed with human fusion partners express hTERT infrequently, not at all, or at sub-optimal levels. This is in accord with the observations of others that human/human hybrid cells formed between TERT-positive and TERT-negative cells are generally TERT-negative (15, 16, 17). Accordingly, Applicants predict that hybrid cells formed between human fusion partner cells and human primary cells such as B-lymphocytes will also suffer from inadequate hTERT expression. Human/human hybrid cells are specific embodiments of the present invention when provided with ectopic expression of hTERT.

a. Fusion Partner Cell Lines

Cell lines suitable for use as fusion partner cell lines in the present invention include mammalian cell lines, such as those of human, murine, or other origin including amphibians, birds, camels, cats, cows, dogs, donkeys, goats, horses, rabbits, rats, sheep, swine, and non-human primates; cell lines of B-lineage, especially myeloma cell lines and lymphoblastoid cell lines; cell lines that are able to grow in vitro; immortal cell lines that express an endogenous telomerase gene; immortal cell lines that express an ectopic telomerase gene; immortal cell lines that do not express a telomerase gene.

b. Fusion Cells

(1) Human B-lineage Cells

In the method of generating hybrid cells that express an ectopic TERT gene and secrete immunoglobulins, preferred fusion cells are cells of B-lineage, and in particular primary B-lymphocytes. B-lineage cells can be obtained from living humans with minimal risk and discomfort by phlebotomy or leukopheresis. B-cells can be obtained from the spleen, obtained either at splenectomy or autopsy, and from tonsils removed by tonsillectomy. Furthermore, B-cells can be isolated from lymph nodes obtained at biopsy indicated for the purposes of diagnosis or cancer staging.

In order to make monoclonal antibodies with particular antigenic specificities, B-cells can be obtained from individuals who have been exposed to antigens of interest (antigens against which monoclonal antibodies are to be produced). Specific embodiments of the present invention therefore include forming hybridomas by fusing fusion partners as described herein and B-cells from people who have been or are currently affected by an infectious disease, or who have been immunized or otherwise exposed to components of the infectious agent such as a vaccine. When isolated and purified, such antibodies can be used to provide passive immunity to a person affected with an illness prior to the time when his/her own immune system has generated antibodies.

For instance, in infants the natural decay of maternal antibodies to Haemophilus influenzae engenders a significant risk of contracting meningitis due to the organism. An intravenous infusion of antibody may either reduce the risk of contracting the disease or help to facilitate the recovery of affected infants.

Disease-specific antibodies may also be useful in treating infections that for which there is currently no satisfactory therapy. For example, antibiotics for treating vancomycin-resistant Enterococcus infections are few and of limited efficacy. They may work better when given in conjunction with a specific antibody preparation.

Antibodies of the present invention may be also be useful for patients who lack the ability to effectively make their own antibodies, either due to a specific immunodeficiency syndrome, immunosuppressive medications, general physical deterioration, or the effects of having undergone a bone marrow transplant.

Pathogen-specific antibodies can serve as primary prophylaxis in the event of exposure to a pathogen, such as a virus (e.g., the Human Immunodeficiency Virus (HIV), influenza virus, hepatitis virus) a parasite (e.g., malaria parasite) a bacterium (e.g., salmonella, E.coli) or a fungus (e.g., Candida). For example, administration of antibodies directed at the HIV or a component thereof (e.g. HIV gp41) may be an effective means of preventing the establishment of HIV infection in a person who has been or may become exposed to the virus.

Antibodies made by hybrid cells of the present invention, created with B-cells from individuals who have been exposed to infections or infection-related antigens, will have broad utility. In particular, they will make it possible to passively immunize a person against any antigens by providing him with antibodies directed against the antigens. Hybrid cells that produce such antibodies and the antibodies they produce that bind to such antigens are subjects of this invention.

Further, patients with cancer, including solid tumors and malignant hematological diseases such as leukemia and lymphoma, may develop antibodies against their own malignancies. When isolated and purified, such antibodies may be effective as anti-tumor therapies. Monoclonal antibodies have been shown to be efficacious against some non-Hodgkin's lymphomas and some breast cancers. They can also be useful for the development of diagnostic tests, either detecting the presence of a tumor antigen in the blood of an individual or, through conjugation with a radioactive or other element that allows the antibody to be visualized in the body by radionuclide or other scanning modalities. Hybrid cells of the present invention that bind antigens expressed by malignant cells can be produced by fusing B-cells from patients with cancer with fusion partners. Such hybrid cells and the monoclonal antibodies they produce are specific embodiments of the present invention.

It is also possible to create hybridomas with B-cells from patients with autoimmune diseases syndromes that are associated with the development of auto-antibodies, such as rheumatoid arthritis and systemic lupus erythematosis. The development of such antibodies may be helpful in understanding the pathogenesis of autoimmune diseases and aid the identification of specific auto-antigen binding sites that may serve as useful drug targets. Such hybrid cells and the monoclonal antibodies they produce are further embodiments of this invention.

It may be of interest to generate monoclonal antibody producing cells from individuals who have not had recent exposure to specific antigens, or who have been exposed to only a small amount of antigen, and may therefore possess relatively few B-cells with the desired antigen specificity. In order to increase the proportion of B-cells in such a population that have the desired specificity proliferation of B-cells can be stimulated in vitro in the presence of the desired antigen. Hybrid cells formed with B-cells stimulated with specific antigens and the antibodies they produce are specific embodiments of the present invention.

(2) Non-human B-lineage Cells

Many potentially useful antibodies may be difficult to obtain due to the ethical problem of immunizing humans with antigens that may pose them harm. For instance, it would be difficult to generate human monoclonal antibodies as a specific antidote for neurotoxins such as Sarin, because it would be unethical to inoculate a human with neurotoxin-related antigens. Some non-human primates may generate antibodies sufficiently similar to human antibodies that the antibodies would not be detected as foreign by the human immune system. Such primates can be inoculated with the neurotoxin antigens. In an embodiment of the present invention, B-lymphocytes from the inoculated animals are fused to fusion partners in order to create hybrid cells that produce antibodies that bind to the neurotoxin antigens. Such antibodies can be used as an antidote for the toxin. If non-human primates prove to produce antibodies that are compatible with the human immune system the ability to make medically useful antibodies using the present invention will be greatly enhanced.

The present invention is also useful to produce antibodies against a drug that is toxic if over-dosed and not inherently immunogenic by itself. Two examples are acetaminophen and digoxin. A non-human primate can be immunized with these drugs in an immunogenic form, such as conjugated to a hapten and mixed with Freund's Adjuvant. Following a course of immunization, the animal's B-cells could be used within the scope of the present invention to produce hybrid cells that generate antibodies directed against the drug antigens. Such antibodies could be used as an antidote to overdose of the drugs.

The ability to make monoclonal antibodies from non-human animals makes it possible to develop veterinary therapeutics. For these reasons, the present invention can be used to form monoclonal antibody-secreting hybrid cells with B-cells from non-human mammals, pets, or other animals of commercial or other interest. Antibody-secreting hybrid cells can be produced using B-cells from mammals, pets, and other animals such as fish, birds, camels, cats, cows, dogs, donkeys, goats, horses, mice, rabbits, rats, sheep, swine, crustaceans, and non-human primates.

c. Telomerase Genes

A wide variety of telomerase genes (DNA encoding functional telomerase) can be used to produce hybrid cells that ectopically express telomerase.

Any telomerase gene (wild-type, mutated, truncated or otherwise altered) can be used that, when expressed ectopically in hybrid cells, facilitates the production of hybrid cells that produce antibodies.

For example, any telomerase gene (wild-type, mutated, truncated or otherwise altered) can be used that, when expressed in the hybrid cells, confers upon the hybrid cells one or more phenotypic features selected from the following: improved rate of growth; improved quantity of Ig expression by hybrid cells; improved stability of Ig expression; improved ability to be cloned by limiting dilution. For example, telomerase genes from humans, mice, non-human primates, non-primate mammals, or other organisms can be used. Specifically, a telomerase gene derived from an organism selected from the group consisting of humans, amphibians, birds, camels, cats, cows, dogs, donkeys, goats, horses, mice, rabbits, rats, sheep, swine, non-human primates, crustaceans, protozoa and yeasts can be used. Particularly preferred telomerase genes are human and non-human primate telomerase genes. As the term is used herein, a telomerase gene is derived from an organism or animal if it has been obtained or isolated from that organism or has the same or substantially the same sequence as the endogenous telomerase gene of the organism and has been made or synthesized (e.g., by recombinant DNA methods, synthetic methods). For example, a telomerase gene derived from a human can be obtained from a human cell or can be produced to have the same or substantially the same sequence as the human telomerase gene (e.g., hTERT). In a specific embodiment, the human telomerase gene is used.

As described herein, hTERT expression improved the ability of the SP2/0 cells to form fusions with murine B-lymphocytes and to survive high-density culture conditions. It follows that any telomerase gene (wild-type, mutated, truncated or otherwise altered) can be used that, when expressed ectopically in fusion partner cell lines, confers upon the fusion partner cell lines reduced density-dependent cell death or improved hybrid cell formation as reflected in increased hybrid cell colony size or number. For example, telomerase genes from humans, mice, non-human primates, non-primate mammals, or other organisms can be used. Specifically, a telomerase gene derived from an organism selected from the group consisting of humans, amphibians, birds, camels, cats, cows, dogs, donkeys, goats, horses, mice, rabbits, rats, sheep, swine, non-human primates, crustaceans, protozoa and yeasts can be used. Particularly preferred telomerase genes are human and non-human primate telomerase genes. As the term is used herein, a telomerase gene is derived from an organism or animal if it has been obtained or isolated from that organism or has the same or substantially the same sequence as the endogenous telomerase gene of the organism and has been made or synthesized (e.g., by recombinant DNA methods, synthetic methods). For example, a telomerase gene derived from a human can be obtained from a human cell or can be produced to have the same or substantially the same sequence as the human telomerase gene (e.g., hTERT). In a specific embodiment, the human telomerase gene is used.

d. Ectopic Expression of Telomerase

A variety of methods can be used to cause the ectopic expression of telomerase in hybrid cells. For example, telomerase-encoding DNA can be introduced into hybrid cells by a variety of methods. An ectopic telomerase gene can be introduced at any time in the process of cell fusion. It can be introduced into the fusion cells or the fusion partner cell line prior to fusion. It can be introduced into the hybrid cells as they form during the cell fusion, or it can be introduced into the hybrid cells after they have been formed by cell fusion.

In a particular embodiment, an ectopic telomerase gene is introduced into a fusion cell line prior to its use in the creation of hybrid cells. Applicants have shown this to be an efficacious and efficient means of introducing an ectopic telomerase gene into hybrid cells.

Methods for introducing a telomerase gene into the hybrid cells or into cells prior to or during cell fusion include the following: introducing telomerase-encoding DNA in plasmid vectors or other appropriate constructs into the cells using transfection (with calcium phosphate, proprietary lipid-compounds, or other methods), electroporation, microprojectile bombardment and any other method by which telomerase-encoding DNA can be introduced into cells; or by using RNA or DNA viruses, such as retroviral vectors and adenoviral vectors, to infect the cells. A telomerase gene that has been introduced by these or other methods is an ectopic telomerase gene and its expression would constitute ectopic expression of telomerase. Within the hybrid cell the telomerase gene can exist as an integrated provirus, or in DNA that exists independent of the primary genome of the cell, such as in an episome or in a double minute chromosome.

As described in Example 2, retroviral gene transfer is useful to introduce telomerase-encoding DNA into a fusion partner cell line to produce a fusion partner cell line in which telomerase is ectopically expressed.

The present invention relates to methods and DNA constructs to provide a hybrid cell with an ectopic telomerase gene that is in such a form that it will be expressed in the hybrid cell, for instance, in a DNA construct containing DNA sequences that cause telomerase gene expression. Such sequences may be an enhancer element, a promoter element, or both an enhancer element and a promoter element. The enhancer element can be from a wide variety of sources, including, but not limited to, viral enhancers, eukaryotic enhancers, prokaryotic enhancers and synthetic enhancers. The same is true of the promoter element, which can be a viral promoter, an eukaryotic promoter, a prokaryotic promoter or a synthetic promoter. Examples of such sequences include retroviral LTR sequences or the phosphoglycerate kinase (PGK) promoter.

Cells that have acquired and express an ectopic TERT gene can be identified by single cell cloning and analyzing the cloned cells for the presence of ectopic TERT mRNA using RT-PCR with primers specific for the ectopic gene.

Also the subject of this invention are methods and DNA constructs to replace, counteract, or inhibit the effect of DNA sequences that repress or fail to activate the expression of the endogenous telomerase gene. Lack of expression of the endogenous telomerase gene is likely to be mediated by such DNA sequences; therefore, constructs of the present invention are useful to overcome the effects of these DNA sequences and cause expression of the endogenous telomerase gene in hybrid cells. For instance, DNA sequences that prevent telomerase gene expression in the hybrid cells are replaced with DNA sequences that are permissive of telomerase gene expression. Such sequences may be an enhancer element, a promoter element, or both an enhancer element and a promoter element. The enhancer element can be from a wide variety of sources, including, but not limited to, viral enhancers, eukaryotic enhancers, prokaryotic enhancers and synthetic enhancers. The same is true of the promoter element, which can be a viral promoter, an eukaryotic promoter, a prokaryotic promoter or a synthetic promoter. Examples of such sequences include retroviral LTR sequences or the phosphoglycerate kinase (PGK) promoter. An endogenous telomerase gene that has been modified such that it is expressed in hybrid cells is an ectopic telomerase gene. Expression of such a gene constitutes ectopic expression of telomerase and is an embodiment of the present invention.

Cells that have undergone alteration of their DNA in order to cause such ectopic TERT expression can be identified by single cell cloning and analyzing genomic DNA of the cloned cells for the presence of the altered DNA sequences using PCR with primers specific for the altered DNA sequences.

In another embodiment, a telomerase gene is inserted by homologous recombination or other methods into a site within the genome that would ensure its expression in hybrid cells. For example, DNA that is sufficiently homologous to the murine rosa or GAPDH locus can be used to mediate insertion of a telomerase gene at those sites. Such a telomerase gene is an ectopic telomerase gene and its expression would constitute ectopic expression of telomerase. A wide variety of methods to mediate such an insertion into the genome of mammalian cells are known to those of skill in the art.

Cells that have integrated an ectopic telomerase gene into the genome of a cell can be identified by sing le cell cloning and analyzing genomic DNA of the cloned cells for the presence of the ectopic telomerase gene using PCR with primers specific for the altered DNA sequences. Expression of the ectopic gene can be confirmed with RT-PCR.

The c-myc oncogene, NFκB, and the estrogen receptor have been shown to be potent activators of telomerase expression, and p53 and Mad protein expression have been associated with transcriptional repression of the telomerase gene. Enforcing hTERT expression through the ectopic expression of hTERT transcriptional activators such as c-myc, NFκB, and the estrogen receptor or through inhibitors of hTERT transcriptional repressors such as Mad and p53 fall under the definition of ectopic TERT expression as used herein and are specific embodiments of the present invention.

Cells that ectopically express regulators of telomerase gene transcription can be identified by single cell cloning and analyzing protein or mRNA of the cloned cells for the presence of the products of the ectopically expressed regulators using Western Blotting or RT-PCR. Expression of the ectopic telomerase gene can be confirmed with RT-PCR.

TERT-containing and TERT-regulating gene constructs may also contain genes for selectable markers (products that permit identification of cells of interest, such as antibiotic resistance genes and chromogens) in order to identify cells that have taken up the ectopic gene. If the selectable marker gene encodes antibiotic resistance (e.g., puromycin or G418 resistance), cells that have not taken up the ectopic gene do not grow in the presence of the antibiotic. Genes encoding chromogens such as Green Fluorescent Protein (GFP), Blue Fluorescent Protein (BFP), Red Fluorescent Protein (RFP) and beta-galactosidase can serve a similar role. Cells that have taken up the ectopic gene can be selected on the basis of their expression of the chromogen.

In an embodiment, a DNA construct comprises sequences homologous to the endogenous TERT promoter that flank sequences of a constitutively active promoter, such as the PGK promoter. Such a construct can also contain a selectable marker gene, such as the gene that confers resistance to the antibiotic puromycin, to facilitate identification of cells that incorporate the DNA construct. The DNA construct can be, for example, a plasmid or a viral vector and the DNA can be linear or circular in configuration. The homologous sequences in the DNA construct facilitate recombination in the TERT promoter, replacing DNA sequences that mediate TERT transcriptional repression with DNA sequences that activate TERT expression in hybrid cells. The construct can also include further components, such as a plasmid backbone or selectable marker(s). The identification of successful recombinant cells can be facilitated by culturing cells with an antibiotic such as puromycin; non-recombinant cells do not survive in the presence of the antibiotic. In hybrid cells that have undergone such a modification of their endogenous hTERT regulatory sequences the PGK promoter directs ectopic expression of the endogenous telomerase gene; such a telomerase gene comprises an ectopic telomerase gene. A hybrid cell possessing such an ectopic telomerase gene and the DNA constructs used to create such a hybrid cell are specific embodiments of the present invention.

In an embodiment, a DNA construct comprises sequences that are homologous to chromosomal DNA that is transcriptionally active in hybrid cells flanking a TERT cDNA. Such a construct can also contain a selectable marker gene, such as the gene that confers resistance to the antibiotic puromycin, to facilitate identification of cells that incorporate the DNA construct. The DNA construct can be, for example, a plasmid or a viral vector and the DNA can be linear or circular in configuration. The homologous sequences in the DNA construct facilitate recombination with the chromosomal DNA, thus incorporating the TERT cDNA into a chromosomal region that directs TERT expression in hybrid cells. The construct can also include further components, such as a plasmid backbone or selectable marker(s).

The identification of successful recombinant cells can be facilitated by culturing cells with an antibiotic such as puromycin; non-recombinant cells do not survive in the presence of the antibiotic. In hybrid cells that have undergone such a modification the ectopic expression of hTERT is determined by chromosomal DNA sequences that flank the ectopic hTERT cDNA; such a telomerase gene is an ectopic telomerase gene. A hybrid cell possessing such an ectopic telomerase gene and the DNA constructs used to create such a cell are specific embodiments of the present invention.

e. A Method of Producing Hybrid Cells that Express an Ectopic Telomerase Gene

The present invention relates to a method of producing a hybridoma, comprising fusing a mammalian cell line cell with a fusion cell, under conditions appropriate for production of hybridomas, thereby producing a hybridoma, combined with the introduction of an ectopic telomerase gene. Methods and conditions for producing hybridomas are well known to those of skill in the art and are routine. They are described, for example, in a published laboratory manual (2). The telomerase gene is of human or other origin and can be introduced before fusion (into the fusion partner cell or the fusion cell), during fusion, or after fusion into the newly formed hybrid cell. The telomerase gene is ectopic and capable of expression in the hybrid cells. As described above, the fusion partner cell line used can be, for example, a human cell line or a murine cell line (e.g., a myeloma cell line) and the fusion cell can be a human B-cell or a non-human B-cell, as would exist in an antigen- or mitogen-stimulated human peripheral blood mononuclear cell population. As described above in the context of the cell lines and the introduction of an ectopic telomerase gene, at least one selectable marker gene or at least one gene that encodes a chromogenic protein can be expressed, but is not required. Hybridomas produced by these methods are also the subject of this invention.

f. A Method of Producing Antibodies

Also the subject of this invention is a method of producing monoclonal antibodies that comprises: fusing a fusion partner cell with a fusion cell, under conditions appropriate for hybridoma formation, wherein the fusion cell is a B-lineage cell; introducing an ectopic telomerase gene into one of the cells before, during, or after the fusion process, thereby producing hybridomas that ectopically express telomerase; and maintaining the hybrid cells under conditions appropriate for production of antibodies by the hybridomas, whereby antibodies are produced. Methods and conditions for producing hybridomas and maintaining the hybridomas in order for them to produce monoclonal antibodies are well known to those of skill in the art. Hybridomas of the present invention ectopically express telomerase. Hybridomas can be, for example, of murine, human, or of combined murine/human origin.

The invention also relates to a method of producing human antibodies, comprising: (a) fusing a fusion partner cell with a human B-lineage cell, under conditions appropriate for hybridoma formation (b) introduction of an ectopic human telomerase gene before, during, or after the fusion process (c) thereby producing hybridomas that ectopically express human telomerase and (d) maintaining hybridomas produced in (a) under conditions appropriate for production of antibodies by the hybridomas, whereby human antibodies are produced. Hybridomas of the present invention ectopically express human telomerase. Hybridomas can be, for example, of murine, human, or of combined murine/human origin. Antibodies produced by this method are derived in full or in part from the human B-lineage cell and will therefore be human antibodies. Antibodies produced by this method wherein the fusion partner and the fusion cell are of human origin are fully human antibodies as defined herein.

g. A Method of Producing Human Antibodies of Interest

In one embodiment, this invention is a method of producing a hybridoma that produces antibodies that bind antigens expressed by a malignant cell. The method comprises producing hybrid cells that ectopically express a telomerase gene and produce antibodies that bind to antigens expressed by a malignant cell by fusing a fusion partner cell that ectopically expresses telomerase with a fusion cell of B-lineage derived from a human affected by a malignant disease, under conditions appropriate for formation of hybridomas, whereby a hybridoma that expresses an ectopic telomerase gene and produces antibodies that bind antigens expressed by the malignant cell is produced. Alternatively, an ectopic telomerase gene can be introduced in the fusion cell prior to fusion, or can be introduced into the hybrid cell during or after the formation of the hybrid cell. The malignant cell can be, for example, a cell from a solid malignant tumor or a hematopoietic tumor. The solid tumor can be, for example, a gastrointestinal tumor, a breast tumor, a kidney tumor, a brain tumor, a liver tumor, a stomach tumor, a lung tumor, a pancreatic tumor, a tumor of the reproductive systems, a prostate tumor, an eye tumor, a skin tumor, a melanoma, adenomas, or pre-malignant lesions such as adenomas, polyps, dysplasias, in situ carcinomas, and intra-epithelial neoplasms. The hematopoietic tumor can be, for example, a leukemia, lymphoma, or myeloma, or pre-malignant conditions such as myelodysplastic syndromes.

In one embodiment, this invention is a method of producing a hybridoma that produces antibodies that bind antigens expressed by a pathogens. The method comprises producing hybrid cells that ectopically express a telomerase gene and produce antibodies that bind to antigens expressed by a pathogen by fusing a fusion partner cell that ectopically expresses telomerase with a fusion cell of B-lineage derived from a human affected by the pathogen, under conditions appropriate for formation of hybridomas, whereby a hybridoma that expresses an ectopic telomerase gene and produces antibodies that bind antigens expressed by the pathogen is produced. Alternatively, an ectopic telomerase gene can be introduced in the fusion cell prior to fusion, or can be introduced into the hybrid cell during or after the formation of the hybrid cell. The pathogens against which antibodies are produced by the present method include, but are not limited to, RNA viruses, DNA viruses, bacteria, intracellular parasites, fungi, helminths and protozoa.

RNA viruses against which antibodies can be produced by the present method include, but are not limited to, members of RNA virus families such as Picornaviridae, Calciviridae, Togaviridae, Flaviviridae, Coronaviridae, Rhabdoviridae, Filoviridae, Paramyxoviridae, Orthomyxoviridae, Bunyaviridae, Arenaviridae, Reoviridae and Retroviridae. DNA viruses against which antibodies can be produced by the present method include, but are not limited to, members of DNA virus families such as Hepadnaviridae, Parvoviridae, Papovaviridae, Adenoviridae, Herpesviridae and Poxviridae and Hepatitis.

Bacteria against which antibodies can be produced by the present method include, but are not limited to, gram-positive cocci, gram positive bacilli, gram-negative bacteria, anaerobic bacteria, organisms of the families Actinomycetaceae, Bacillaceae, Bartonellaceae, Bordetellae, Captophagaceae, Corynebacteriaceae, Enterobacteriaceae, Legionellaceae, Micrococcaceae, Mycobacteriaceae, Nocardiaceae, Pasteurellaceae, Pseudomonadaceae, Spirochaetaceae, Vibrionaceae and organisms of the genera Acinetobacter, Brucella, Campylobacter, Erysipelothrix, Ewingella, Francisella, Gardnerella, Helicobacter, Levinea, Listeria, Streptobacillus and Tropheryma. Intracellular parasites against which antibodies can be produced by the present method include, but are not limited to, Chlamydiaceae, Mycoplasmataceae, Acholeplasmataceae, Rickettsiae and organisms of the genera Coxiella and Ehrlichia. The fungi are selected from the group consisting of: Aspergillus, Blastomyces, Candida, Coccidioides, Cryptococcus, Histoplasma, Paracoccicioides, Sporothrix, organisms of the order Mucorales, organisms inducing choromycosis and mycetoma and organisms of the genera Trichophyton, Microsporum, Epidermophyton, and Malassezia. The helminths are selected from the group consisting of: Nematodes, Trematodes or Cestodes.

This invention also relates to a method of producing hybridomas that produce antibodies that bind an antigen such as those in venoms, allergens and other molecules introduced by bites, scratches or other contacts with insects or other animals (e.g., snakes, scorpions, frogs, wasps, bees, mosquitoes, spiders, jellyfish, anemones), comprising forming cell hybrid cells through cell fusions with B-cells from individuals (e.g., humans, non-human primates, and other mammals) who have been exposed to such venoms, allergens or other molecules.

A further embodiment of the present invention is a method of producing hybrid cells in which the fusion cells are B-cells from mammals (e.g., humans) who have been or are affected by an autoimmune disease. Such hybrid cells produce immunoglobulin proteins (antibodies) that are reactive with self-antigens and may bind protein, DNA, RNA, lipid, carbohydrate or other antigens, and may be useful for therapeutic or other purposes. In one embodiment such a method comprises fusing a fusion partner cell that ectopically expresses telomerase with a fusion cell that is a B-cell from an individual who is or has been affected by an autoimmune disease.

A further embodiment of the present invention is a method of producing hybrid cells in which the fusion cells are B-cells from mammals (e.g., humans) who have been or are affected by a prion disease (e.g. Creutzfeld-Jacob Disease, kuru, scrapie, bovine spongiform encephalopathy). Such hybrid cells produce immunoglobulin proteins (antibodies) that are reactive with prion antigens. In one embodiment such a method comprises fusing a fusion partner cell that ectopically expresses telomerase with a fusion cell that is a B-cell from an individual who is or has been affected by a prion disease.

A further embodiment of the present invention is a method of producing hybrid cells in which the fusion cells are mammalian (e.g. human) B-cells stimulated in vitro in the presence of antigens, including but not limited to, protein, carbohydrate, nucleic acids, organic compounds, and inorganic compounds for the purpose of creating hybrid cells that produce immunoglobin porteins that are reactive with the stimulating or other antigens.

A further embodiment of the present invention is a method of producing hybrid cells in which the fusion cells are mammalian (e.g. human) B-cells stimulated in vitro in the presence of antigen preparations derived from living organisms, including but not limited to single cells organisms and multi-cellular organisms and products produced by those organisms, for the purpose of creating hybrid cells that produce immunoglobulin proteins that are reactive with the stimulating or other antigens.

5. Fusion Partner Cell Lines that Express an Ectopic Telomerase Gene

In the process of creating hybrid cells that express an ectopic telomerase gene, the ectopic gene can be introduced before, during, or after the process of cell fusion. In one embodiment, an ectopic telomerase gene is introduced into an immortal fusion cell line prior to its use in the creation of hybrid cells (Example 2).

The present invention comprises fusion partners, such as described in section 4a, that ectopically express telomerase, such as described in sections 4c and 4d, and their use in fusions with fusion cells, such as described in section 4b, to produce hybrid cells that express an ectopic telomerase gene, such as described in section 4e, and produce antibodies to antigens of interest, such as described in sections 4f and 4g.

Prior to the work described herein, it was not recognized that ectopic expression of a telomerase gene in an immortal cell line would improve its ability to serve as a fusion partner cell line in fusions with fusion cells. Therefore, any cell line from any tissue or species of origin that expresses an ectopic telomerase gene can be used in the method of the present invention to produce hybrid cells. The resulting hybrid cells and products produced by the cells are the subject of the present invention.

The present invention encompasses immortal mammalian cell lines that ectopically express telomerase and are useful as fusion partner cell lines to produce hybrid cells (hybridomas) in which telomerase is ectopically expressed; hybrid cells in which telomerase is ectopically expressed; antibodies produced by the hybrid cells; DNA constructs useful for producing immortal mammalian cell lines of the present invention and methods of making and using each of the foregoing. In specific embodiments, the cell line is of human or murine origin. The term “a mammalian cell line that ectopically expresses telomerase” includes cell lines/cells that have themselves been modified, as well as progeny and derivatives thereof.

Prior to the work described herein, it was not recognized that ectopic expression of a telomerase gene in an immortal cell line that expresses an endogenous telomerase would be useful or improve the ability of cells to serve as fusion partners. Therefore, any immortal cell line from any tissue or species of origin that expresses an ectopic telomerase gene as well as an endogenous telomerase gene and is intended for use to produce hybrid cells is a subject of the present invention.

The present invention encompasses immortal mammalian cell lines that ectopically express telomerase in addition to an endogenous telomerase gene (also referred to as mammalian cell lines that have been modified to ectopically express telomerase) and their use as fusion partner cell lines to produce hybrid cells (hybridomas) in which telomerase is ectopically expressed; hybrid cells in which telomerase is ectopically expressed; antibodies produced by such hybrid cells; DNA constructs useful for producing mammalian cell lines of the present invention and methods of making and using each of the foregoing. In specific embodiments, the immortal cell line is of human or murine origin. The term “an immortal mammalian cell line that ectopically expresses telomerase in addition to an endogenous telomerase gene” includes cell lines/cells that have themselves been modified, as well as progeny and derivatives thereof.

Once a cell that expresses an endogenous telomerase gene has been engineered to express telomerase from an ectopic gene, there may be little or no additional benefit resulting from expression of the endogenous telomerase gene. Therefore, an embodiment of the present invention is an immortal mammalian cell that expresses telomerase from an endogenous gene, is subsequently modified to express an ectopic telomerase gene, and then is modified or undergoes a change(s) (e.g., during culturing) such that the endogenous gene is no longer expressed or is expressed at lower levels.

The present invention encompasses immortal mammalian cell lines that formerly ectopically expressed telomerase in addition to an endogenous telomerase gene, but have subsequently been modified or have undergone spontaneous change so that they no longer express the endogenous telomerase gene, and their use as fusion partner cell lines to produce hybrid cells (hybridomas) in which telomerase is ectopically expressed; hybrid cells in which endogenous telomerase is not expressed or is expressed at a lower level than the level at which it was expressed formerly and telomerase is ectopically expressed; antibodies produced by such hybrid cells; DNA constructs useful for producing immortal mammalian cell lines of the present invention and methods of making and using each of the foregoing. In specific embodiments, the cell line is of human or murine origin. The term “an immortal mammalian cell line that formerly ectopically expressed telomerase in addition to an endogenous telomerase gene, but have subsequently been modified to that they no longer express the endogenous telomerase gene” includes cell lines/cells that have themselves been modified, as well as progeny and derivatives thereof.

In all of these embodiments, the telomerase gene can be derived from an organism selected from a wide variety of organisms, including, but not limited to, humans, amphibians, birds, camels, cats, cows, dogs, donkeys, goats, horses, mice, rabbits, rats, sheep, swine, non-human primates, protozoa, crustaceans and yeasts.

Specific embodiments of this invention are immortal human B-lineage fusion partner cell lines that express an ectopic hTERT; their use in fusions with primary human B-lymphocytes to produce fully human hybrid cells in which telomerase is ectopically expressed; fully human hybrid cells in which telomerase is ectopically expressed; and fully human antibodies produced by the hybrid cells.

The immortal mammalian cell line, in the specific embodiments described above, has been modified to ectopically express telomerase from DNA sequences permissive of expression in hybrid cells. The sequences may include, for example, an enhancer element, a promoter element, or both an enhancer element and a promoter element. The enhancer element can be from a wide variety of sources, including, but not limited to, viral enhancers, eukaryotic enhancers, prokaryotic enhancers and synthetic enhancers. The same is true of the promoter element, which can be a viral promoter, an eukaryotic promoter, a prokaryotic promoter or a synthetic promoter (a non-naturally occurring or designed promoter).

In another embodiment, genomic DNA sequences in the immortal mammalian cell line that regulate an endogenous telomerase gene are altered to increase, prolong or enhance expression of an endogenous telomerase gene in hybrid cells, resulting in ectopic expression of the gene as defined herein. Alternatively, a telomerase gene is incorporated in the genome of the cell line at a site such that the telomerase gene is under the control of transcriptional regulatory elements that direct expression of the telomerase gene in hybrid cells. Finally, an endogenous telomerase gene is constitutively expressed as a result of the constitutive expression of a transcriptional activator of telomerase gene expression, such as c-myc, NFKB, or the estrogen receptor, or due to the inactivation of inhibitors of telomerase gene transcription, such as Mad and p53.

The cell line in which telomerase is ectopically expressed may have single or multiple copies of the ectopic gene per cell. Increasing the number of copies of the ectopic gene in the fusion partner cell may improve the efficiency with which hybrid cells acquire a copy of the ectopic gene.

Cells that have acquired an ectopic telomerase gene can be identified by single cell cloning and analysis of the cloned cells for the presence of ectopic TERT mRNA (using RT-PCR with primers specific for the ectopic TERT mRNA), or for the altered genomic DNA sequences that ectopically express the telomerase gene (using PCR with primers specific for the altered DNA sequences). Cloned cells can also be analyzed for the presence of the products of ectopically expressed regulators of TERT transcription using Western Blotting or RT-PCR.

In many instances, immortal mammalian cell lines of the present invention express or are modified to express at least one (one or more) gene that encodes a selectable marker, which make it easier to identify cells that express an ectopic telomerase gene. The selectable marker encoded by the gene can be one that confers resistance to a drug, such as resistance to G418, hygromycin, puromycin, bleomycin or another drug. Alternatively, the immortal mammalian cell lines of the present invention can express at least one gene that encodes a chromogenic protein, such as Green Fluorescent Protein, Blue Fluorescent Protein, Red Fluorescent Protein, Yellow Fluorescent Protein or beta-galactosidase. In one embodiment, the identification of immortal mammalian cells that have acquired an ectopic telomerase gene is facilitated by culturing cells with an antibiotic such as puromycin; cells that have not acquired the ectopic telomerase gene do not survive in the presence of the antibiotic.

Any B-lineage cell line, in particular immortal mammalian cell lines and those of murine and human origin, is suitable as a fusion partner cell line within the scope of the present invention, provided it has undergone manipulation that would ensure the expression of an ectopic telomerase gene in hybrid cells formed with the cell line. This includes immortal cell lines of B-cell lineage regardless of whether they also express telomerase from an endogenous gene. Telomerase-positive B-lineage cell lines modified to express an ectopic telomerase are a subject of this invention.

Myeloma cell lines are immortal, malignant B-lineage cell lines. Myeloma cell lines that express an ectopic telomerase gene have not been used previously as fusion partners for the formation of hybrid cells that secrete human antibodies. Myeloma cell lines that express an ectopic telomerase gene and their use in the formation of hybrid cells that express an ectopic telomerase gene are specific embodiments of the present invention.

Lymphoblastoid cell lines are immortal human B-lineage cell lines that have been infected in vivo or in vitro with the Epstein-Barr Virus and have been established in cell culture. As defined herein, lymphoblastoid cell lines also comprise B-lineage cells that express Epstein-Barr Virus antigens. Lymphoblastoid cell lines that express an ectopic telomerase gene have not been used previously as fusion partners for the formation of hybrid cells that secrete human antibodies. Lymphoblastoid cell lines that express an ectopic telomerase gene and their use in the formation of hybrid cells that express an ectopic telomerase gene are specific embodiments of the present invention.

The SP2/0 cell line is an immmortal murine myeloma cell line (a malignant B-lineage cell) that expresses an endogenous murine telomerase gene and is a frequently used as a fusion partner for forming murine hybridomas (18). As described herein, it was modified so that it expresses an ectopic human telomerase gene. The resulting cell line is referred to as SP2/0 MP-hTERT and is a specific embodiment of the present invention. As described below, ectopic expression of the telomerase gene results in improved ability of this cell line to function as a fusion partner in fusions with human and murine B-cells (compared with the ability of the SP2/0 cell line).

A further embodiment of fusion partners of the present invention is primary B-lineage cells that ectopically express telomerase in such a manner that expression is not subject to repression in hybrid cells formed by fusion of such primary B-cell lineage cells with an appropriate fusion cell. Primary B-lineage cells that have not been adapted to continuous growth in vitro express telomerase from their endogenous telomerase genes in a transient fashion when they are stimulated with growth factors or other mitogens (10). This transient telomerase expression is not associated with unlimited replicative potential, perhaps because telomerase expression is transient or because other genetic and epigenetic events required for sustained in vitro growth have not occurred. However, introduction of a constitutively expressed ectopic telomerase gene has contributed to the immortalization of primary T-lineage cells, and would therefore likely contribute to the immortalization of primary B-lineage cells. The ectopic telomerase gene, resistant to repression in hybrid cells, would facilitate the formation of fused cells that express an ectopic telomerase gene. Therefore, primary B-lineage cells that have been engineered to ectopically express a telomerase gene in a manner that is not subject to repression in hybrid cells are a specific embodiment of fusion partners of the present invention.

Fusion partner cells that have undergone genetic or other modifications that improve their effectiveness as fusion partners are also specific embodiments of the present invention. Such modifications could, for instance, improve the rate at which cell fusions form viable hybrids, improve the growth properties of the hybrids, provide or induce the formation of selectable marker genes for the identification of successful hybrids, and improve other properties of the hybrids such as the level of immunoglobulin expression.

Fusion partner cell lines derived from organisms other than humans and old-world primates may lead to the creation of hybrid cells that secrete antibodies modified by Ga1α1-3Gal glycosylation. Humans and old-world primates have native, high-titer antibodies that recognize this antigen that may compromise the ability of antibodies bearing this antigen to be used as medical therapeutics. For instance, immune complexes may form that could lead to premature clearing of antibodies bearing this antigen from the serum. Therefore, immortal mammalian cell lines that are modified so as to be deficient in the enzyme α-1,3-galactosyltransferase are specific embodiments of the present invention.

The hybrid cells that result from fusion of our fusion partner cell lines and fusion cells are specific embodiments of the present invention, as are the antibodies produced by the hybrid cells.

The present invention is illustrated by the following examples, which are not intended to be limiting in any way.

Methods and Materials

The following methods and materials were used in the examples that follow.

Cell culture and Cell fusions:

The SP2/0 and HS Sultan cell lines (ATCC) were maintained in RPMI with 10% heat-inactivated fetal calf serum, with penicillin/streptomycin. 293T cells (ATCC) were maintained in DME with 10% heat-inactivated fetal calf serum, with penicillin/streptomycin. Antibiotic selection was in the presence of 0.5 micrograms/ml puromycin. Fusions were performed with polyethylene glycol and selection in HAT medium (GIBCO) following standard techniques (2).

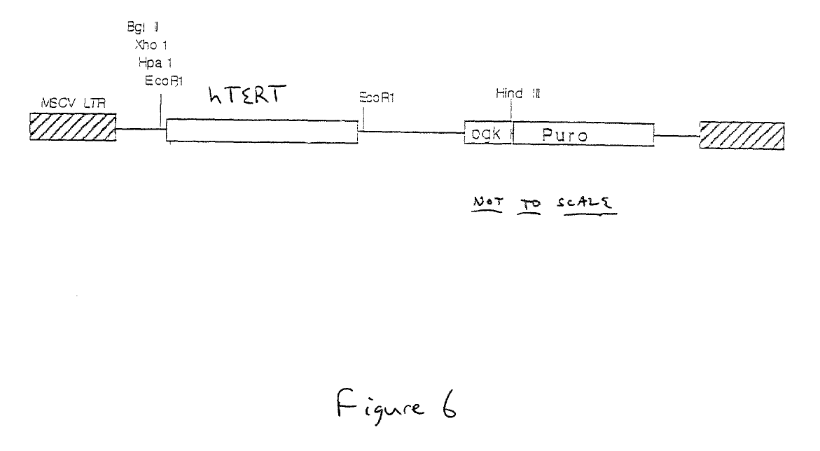

Retroviral infection. A retrovirus containing an hTERT cDNA was constructed using standard recombinant DNA techniques. Retrovirus-containing supernatants were produced in 293T cells using the following variation on standard techniques: 500,000 cells were plated on 6 cm dishes in 4 ml culture medium. The following day, 1 microgram retroviral vector DNA (MSCV Puro hTERT, FIG. 6) and 1 microgram of packaging plasmid (pCL AMPHO (19)) were co-transfected using FUGENE (Roche) using standard protocols. The next day, the medium was replaced with 4 ml RPMI medium. Recipient cells (SP2/0) were seeded in 12 well dishes: 500,000 cells in 2 ml medium. The following day, the supernatant from the transfected 293T cells (containing infectious, replication incompetent retroviruses) was filtered through a 45 micron syringe filter, supplemented with 8 micrograms/ml polybrene, and applied to the recipient cells. Cells were spun in a tabletop centrifuge for 90 minutes at 1500 RPM at 32 C. Cells were then moved to 37 C for an additional 90 minutes. The retroviral supernatant was then replaced with fresh culture medium. The spin/infection process was repeated 24 hours later. The day after the second infection, the cells were brought up to 5 ml total volume with fresh medium. Puromycin (Sigma) selection was initiated after 24 additional hours.

Human peripheral blood mononuclear cells (PBMCs). Human PBMC's were obtained as anonymous discarded samples, consisting of excess cells obtained at leukopheresis from normal human donor who were donating their PBMCs for use in bone marrow transplant patients. The samples were provided to us as anonymous samples, stripped of all patient- and donor-identifying information. The cells were stimulated prior to fusion with pokeweed mitogen using standard techniques.

RT-PCR for hTERT mRNA. 5 micrograms of total RNA was used in a cDNA synthesis reaction using the First-strand cDNA synthesis kit (Pharmacia), with the reverse hTERT and GAPDH primers, each at 2 mM. PCR reactions were run in standard conditions with 2.5 units Taq Polymerase (Perkin Elmer) complexed with Taq Start antibody (Clontech), and 32P-labeled forward primers. Reactions analyzing each mRNA were run separately. Five microliters of cDNA were used in the hTERT and mTERT reactions; one microliter of a 1:400 dilution of the cDNA was used in the GAPDH reactions. PCR reactions were cycled 25 times: 94 C for 30 s, 60 C for 30 s, 72 C for 30 s. Ten microliters of each reaction were analyzed with 8% PAGE, 1× TBE. mTERT primers: AN1 forward (TGAGCGGACAAAACATCC; SEQ ID NO.: 1) AC1 reverse (AGGCTCGTCTTAATTGAGGT; SEQ ID NO.:2) hTERT primers: LT5 forward (CGG AAG AGT GTC TGG AGC AA; SEQ ID NO.:3) and LT6 reverse (LT6 GGA TGA AGC GGA GTC TGG A; SEQ ID NO.:4) (20) GAPDH primers: GAPDH1 forward (GAC CCC TTC ATT GAC CTC AAC: SEQ ID NO.:5) and GAPDH2 reverse primer (CTT CTC CAT GGT GGT GAA GA;SEQ ID NO.:6).

ELISA assays. Human immunoglobulin secretion was analyzed using standard techniques for ELISA assays (2). Wells in standard 96-well ELISA plates were coated with thirty microliters of primary 01 Rabbit anti-human IgG specific for heavy and light chains antibodies from (Southern Biotech) at a concentration of 2 micrograms/ml. The same antibody, conjugated to horseradish peroxidase, was used as a secondary (Southern Biotech)at a 1:3000 dilution in phosphate buffered saline/0.1% bovine serum albumin. Assays were developed using standard techniques with a chromogenic substrate.

TRAP assay. Telomerase catalytic activity was assayed using the TRAP assay following standard techniques (21).

### EXAMPLES

### Example 1

### The Endogenous Telomerase Gene is Repressed in Murine/human hybrid cells.

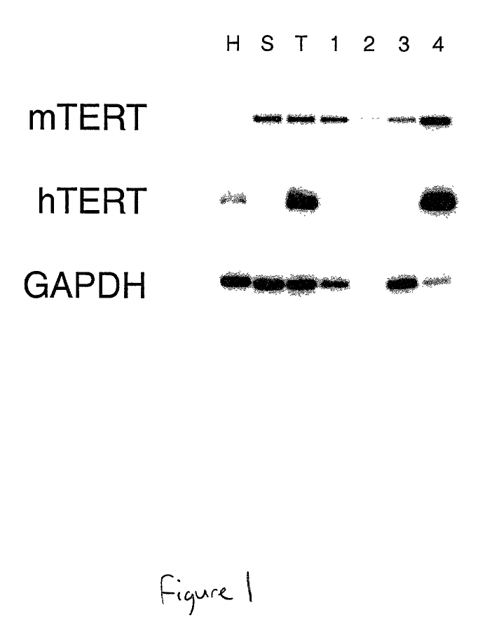

The expression of hTERT and mTERT in hybrid cells formed between primary human cells and murine cells has not been previously reported. Applicants analyzed the expression of the hTERT and mTERT genes in hybrid cell populations using RT-PCR. Each was taken from one of the wells of the original 24-well plate, and therefore may represent a monoclonal or polyclonal population. Data from the two hybrid populations formed from SP2/0 cells and human B-cells are shown in FIG. 1, lanes 1 and 2. These populations express only mTERT and not hTERT. This novel result suggests that hybrids between murine cell line and primary human cells do not express hTERT, but do express mTERT.

One of the two hybrid populations formed between SP2/0 MP-hTERT cells and human B-cells does not express hTERT from either an endogenous or an ectopic hTERT gene, although it does express mTERT (FIG. 1, lane 3) This provides further evidence that such murine/human hybrid cells do not express an endogenous hTERT.

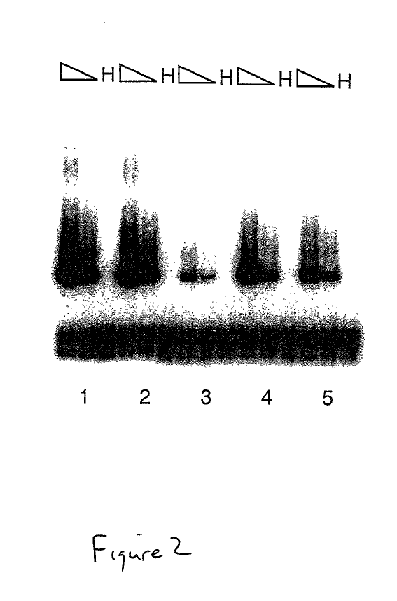

TRAP assays depicted in FIG. 2 reveal that the mTERT in these cell lines is catalytically active (the cells depicted in FIG. 1, lanes 1, 2, and 3 are the same as those depicted in FIG. 2, lanes 2, 3, and 4, respectively).

These results confirm Applicant's original hypothesis that such hybrids do not express hTERT. Furthermore, they suggest that hybrid cells formed between immortal mammalian cell lines and primary human B-cells may benefit from the expression of an ectopic hTERT.

### Example 2

### Creation of a Murine Myeloma Cell Line that Ectopically Expresses Human Telomerase.

Initial experiments made use of the SP2/0 murine myeloma line, which is routinely used for the formation of hybridomas with murine B-lymphocytes in order to produce murine monoclonal antibodies (18). The SP2/0 cell line is resistant to 6-thioguanine, allowing fusions between it and normal cells to be selected by their ability to survive in HAT medium.

Retroviral-mediated gene transfer was used to introduce and ectopically express hTERT in the SP2/0 cell line. An hTERT cDNA was subcloned into the MSCVpuro retroviral vector. This vector contains a viral LTR that directs high level expression of subcloned transgenes, in addition to a puromycin gene driven by the potent PGK promoter. Retroviruses were produced using a protocol that is an adaptation of standard techniques and cells were infected using a centrifugation protocol (see Materials and Methods). A polyclonal population of cells that had been infected and had integrated DNA copies of the retroviral genome into their genomic DNA was selected on the basis of its ability to survive in the presence of puromycin. We created 2 populations of cells, one that expressed the hTERT transgene (SP2/0 MP-hTERT) and a control cell line that expressed vector sequences only (SP2/0 MP).

Expression of the hTERT was confirmed using RT-PCR (see Materials and Methods). Total RNA was prepared from the polyclonal cell populations after they had survived puromycin selection and analyzed for mTERT and hTERT expression using Reverse Transcriptase/PCR (RT-PCR). RNA samples were also analyzed for Glyceraldehyde Phosphate Dehydrogenase (GAPDH) as a positive control for the presence of undegraded RNA. The primers used were specific for either hTERT or mTERT. Mock-RT reactions were also analyzed to control for the possibility of DNA contamination in the RNA samples.

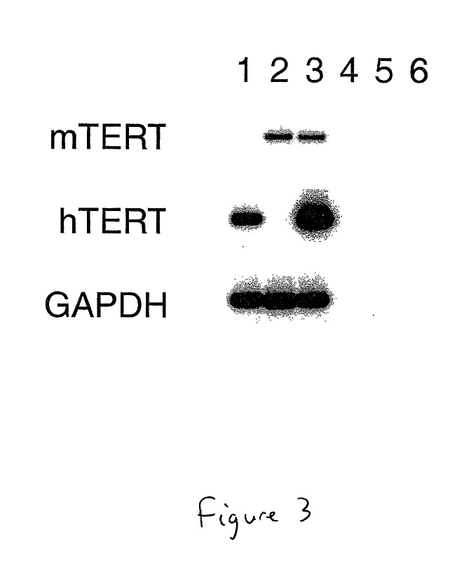

As revealed in FIG. 3, SP2/0 MP cells express only mTERT (lane 2) whereas SP2/0 MP-hTERT cells (lane 3) express mTERT and hTERT. The mock-RT reactions were uniformly negative. The hTERT signal from the SP2/0 MP-hTERT cells is considerably higher than that generated by the HS Sultan human lymphoblastoid cell line (lane 1) indicating the high expression levels characteristic of genes driven by the MSCV retroviral LTR in hematopoietic cells.

In summary, we have created a novel cell line that expresses an ectopic human telomerase gene in addition to an endogenous murine telomerase gene. The cell line is a murine myeloma cell line and the promoter directing expression of the ectopic human telomerase gene is of retroviral origin. The cell line expresses a gene that confers resistance to the drug puromycin and therefore serves as a selectable marker.

### Example 3

### Efficient Creation of Murine/Human Cell Hybrids Expressing an Ectopic Telomerase Gene by First Ectopically Expressing hTERT in a Murine Cell Line.

The present invention is based on Applicants' observation that hybrid cells formed between cell lines and primary human cells benefit from the expression of an ectopic hTERT. The order in which the three components are combined is not essential to the present invention. Applicants considered that the most straightforward way to introduce an ectopic hTERT into the hybrid cells may be to express it in the fusion partner cell line prior to cell fusion.

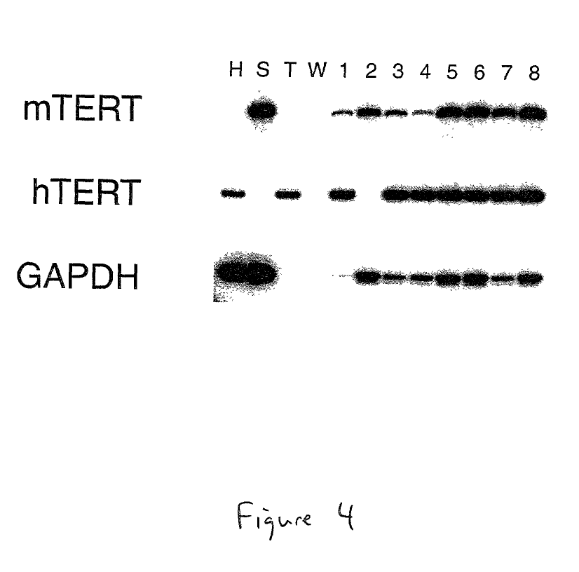

Expression of an ectopic hTERT was assessed in a series of hybrid cell populations formed in the 24-well dishes from fusion of the SP2/0 MP-hTERT cell line with human B-cells. As seen in FIG. 4, all but one of 8 of the populations expressed hTERT (lanes 1-8; n.b. all but the cells in lane 8 were positive for human Ig expression). The levels are comparable to those seen in the SP2/0 MP-hTERT line and much higher than the expression levels of endogenous hTERT in cultured cell lines. Taken together with the observation from Example 1 that hybrids between SP2/0 cells and human B-lymphocytes are negative for hTERT, this result suggests that the expressed hTERT is an ectopic hTERT contributed by the SP2/0 MP-hTERT cells. Furthermore, this result suggests that ectopic expression of an hTERT in a fusion partner cell line prior to cell fusion is a reliable way to create hybrid cells that express an ectopic hTERT.

### Example 4

### Correlation of Ectopic hTERT Expression with Immunoglobulin Expression and Favorable Growth Characteristics of Murine/Human Cell Hybridomas.

Expression of hTERT and mTERT in two hybrid cell populations that were formed with the SP2/0 MP-hTERT fusion partner is depicted in FIG. 1 (lane 3 (Ig negative) and lane 4 (Ig positive). Both cell lines grow rapidly in culture. One of the populations was positive for Ig, and one was negative. This reveals a correlation between expression of an ectopic hTERT and expression of human Ig: only the hTERT-positive cell line expressed Ig. The Ig-negative line did not express hTERT from either the ectopic or an endogenous promoter.

Applicants also compared the growth characteristics of two Ig-positive hybrid cell populations, one hTERT-negative and one hTERT-positive (FIG. 1, lanes 2 and 4, respectively). The one without hTERT grew very slowly in culture, with a doubling time of over a week, whereas the one with hTERT grew well in culture with a doubling time of less than 2 days. This is consistent published reports that Ig-positive human hybrid cells created with the presently available methods grow poorly in culture. Furthermore, this result suggests suggests that hTERT can provide human Ig-producing hybrid cells with phenotypic advantages in addition to those provided by mTERT.

The hTERT-positive, human Ig-expressing cell population (FIG. 1, lane 4) continued to express Ig following 2 months of growth as a tumor in an immunocompromised mouse, indicating superlative stability of Ig expression (data not shown).

Taken together, these results indicate that hTERT expression correlates with the combined phenotypes of excellent in vitro growth and stable human Ig expression. Because all of the hybrid cell populations express mTERT, these results also indicate that hTERT has phenotypes distinct from and superior to those of mTERT. Finally, these results suggest that the poor growth and loss of Ig expression previously seen with hybrid cells formed with human B-lymphocytes represents primarily a deficiency of phenotypes that can be conferred by ectopic hTERT expression.

### Example 5

### Ectopic Human Telomerase Gene Expression Improves the Ability of a Cell Line to Form Immunoglobulin-Expressing Cell Hybrids with Human B-Lymphocytes.

The ability of the SP2/0 and the SP2/0 MP-hTERT cell lines to form productive cell fusions which with human B-lymphocytes were compared. Cells were fused using standard protocols with with pokeweed mitogen-stimulated human peripheral blood mononuclear cells, which are enriched for B-cells, and distributed into 24 well plates in the presence of HAT medium (Ausubel, #4). The SP2/0 and the SP2/0 MP-hTERT cell lines were equivalent in their ability to establish hybrid cells. Out of 24 wells, each fusion had 21 wells positive for hybridoma formation, and all were initially positive for human immunoglobulin (Ig) by ELISA assay. However, by 4 weeks, 5/21 of the SP2/0 wells had lost human Ig expression whereas none of the SP2/0 MP-hTERT wells had done so. In addition, we noted a strong qualitative trend of higher levels of Ig expression in the SP2/0 MPT hybridomas. These results indicate that ectopic hTERT expression enables a murine fusion partner cell line to create hybrid cells that have increased levels and stability of human Ig expression, relative to cells created using the presently available methods.

Taken together with the findings of Example 3, that fusion partners expressing an ectopic hTERT efficiently confer ectopic hTERT expression upon hybrid cells derived from them, these results suggest that ectopic hTERT expression increases the level and stability of human Ig expression in hybrid cells. They further establish the utility of ectopic telomerase expression in the establishment and maintenance of hybrid cells that express human antibodies.

The ELISA assay used is specific for human Ig and does not detect murine Ig. Therefore, the immunoglobulins produced by the hybrid cells could not have been derived from the genome of the SP2/0 MP-hTERT cells. Instead, they are native human antibodies derived from the human B-lymphocytes.

### Example 6

### Assessment of Cloning of Hybrid Cells by Limiting Dilution.

The ability to be clone hybrid cells by limiting dilution is essential for the creation of monoclonal cell lines and the monoclonal antibodies they produce. Applicants tested hybrid cells, made with and without ectopic hTERT, for their ability to be cloned by limiting dilution. Following cell fusion, 6 wells containing hybrid cells formed with either the SP2/0 and SP2/0 MP-hTERT cells and human B-cells were plated at a density of 1, 5, and 10 cells per well. In Table 1 the number of wells positive for growth at 6 weeks is reported, in addition to the proportion of wells that are positive for human Ig production. All six of the SP2/0 MP-hTERT wells contained hybridomas that could be cloned by limiting dilution; only three of the SP2/0-derived hybridomas could be cloned. Furthermore, three of the SP2/0 MP-hTERT wells contained hybridomas that could be cloned at a density of one cell per well, whereas only one of the SP2/0 wells contained hybridomas that could be cloned at a density of either one or five cells per well.

The clones were tested for human Ig using ELISA. Only two of the six wells bearing hybridomas generated with the SP2/0 cell line contained cells that expressed human Ig following limiting dilution (Table 2). In contrast, five of the six wells containing SP2/0 MPT hybridomas gave rise to clones that expressed human Ig.

In summary, the expression of an ectopic hTERT improved the ability of hybrid cells to be cloned by limiting dilution. Furthermore, ectopic hTERT improved the ability of the hybrid cells to sustain Ig expression during the process of cloning. The monoclonal cells thus created produce native human monoclonal antibodies.

### Example 7

### Ectopic Human Telomerase Gene Expression Reduces Density-Dependent Cell Death of the SP2/0 Murine Myeloma Cell Line.

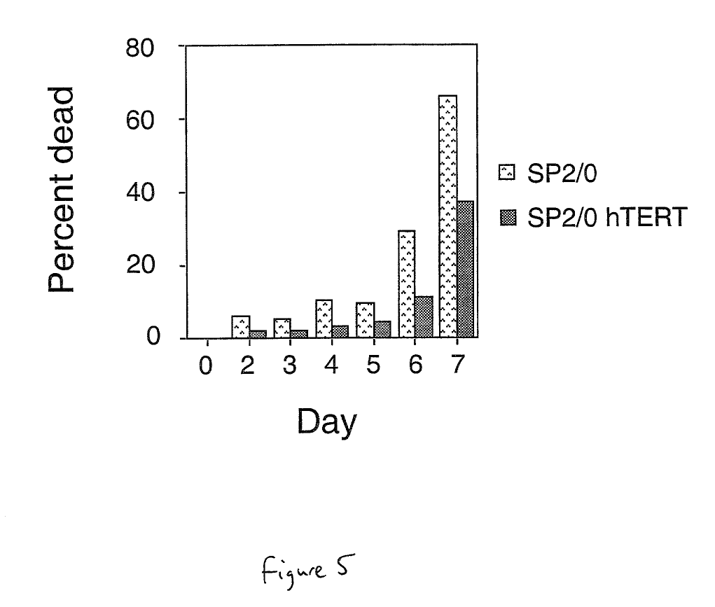

The SP2/0 cell line is susceptible to spontaneous cell death when cultured at high density. Applicants compared the ability of the SP2/0 and of the SP2/0 MP-hTERT cells to survive high density culture. SP2/0 and SP2/0 MP-hTERT were grown to confluence and maintained without splitting for 5 days. Aliquots of the cultures were tested daily for the ability to exclude the Trypan Blue dye, an indicator of cell viability; the proportion of dead cells was calculated for SP2/0 cells and SP2/0 hTERT. Ectopic expression of a human telomerase gene provided SP2/0 cells with resistance to density-dependent cell death (FIG. 5). By day 6, 29% of the SP2/0 cells were dead compared with 11% of the SP2/0 MPT (hTERT-expressing) cells. By day 7, the proportion of dead cells had increased to 66% and 37%, respectively.

In summary, the expression of an ectopic human telomerase gene in a cell that expresses an endogenous murine telomerase gene is useful in that it provides the cell with improved survival under conditions of high-density cell culture.

### Example 8

### Ectopic hTERT Expression Improves the Ability of a Murine Cell Line to Form Murine/Murine Hybrid Cells.

SP2/0 cells express mTERT. Therefore, we reasoned that the survival benefit hTERT confers upon SP2/0 MP-hTERT cells may not be derived from synthetic activity at the mouse telomeres. Instead, it may result from an anti-apoptotic or other survival-promoting activity. Therefore, we assessed of whether hTERT expression would improve the success of murine/murine cell fusions. SP2/0 and SP2/0 MP-hTERT cells were fused with LPS-stimulated murine splenocytes (a cell population consisting substantially of B-cells) and distributed into two 24-well plates in HAT selection medium following standard protocols. The SP2/0 MP-hTERT cells formed productive fusions at a greater rate than the SP2/0 cells. All 48 of the wells containing SP2/0 MPT cells grew hybridoma clones, compared to 33 of the 48 wells containing SP2/0 cells.

Sixteen of the wells, F1-F8 and A1-A8, were subsequently cloned by limiting dilution. The number and size of clones that grew following dilution are shown in Table 2. The number of hybridomas growing in each well was greater in the SP2/0 MP-hTERT fusion, as were the sizes of the individual clones. The SP2/0 MP-hTERT fusion gave 43 large colonies and 95 overall, compared with the SP2/0 fusion that gave 15 large colonies and 48 overall.

Thus, ectopic human telomerase gene expression in a murine myeloma cell line that expresses an endogenous mTERT is useful in that it provides the cell line with an improved ability to form hybridomas with murine B-lymphocytes, as reflected by the quantity and size of hybrid cell colonies formed. The increased efficiency in establishing clones and the overall increase in clone size engendered by ectopic hTERT expression are superior to those of the presently available methods of creating murine hybridomas. Therefore, the present invention is also an improvement in the production of murine monoclonal antibodies.

TABLE 1
Cloning by limiting dilution and immunoglobulin expression
of hybridomas formed between murine SP2/0 and SP2/0
MP-hTERT with human B-lymphocytes.

| | 1 cell/well | | 5 cells/well | | 10 cells/well | |
|---|---|---|---|---|---|---|
| Density: | | # positive | | # positive | | # positive |
| Well | # clones | # tested | # clones | # tested | # clones | # tested |

SP2/0

| | | | | | | |
|---|---|---|---|---|---|---|
| 1S | 39 | 3/17 | 87 | 8/25 | ˜113 | 2/3 |
| 2S | 0 | ND | 0 | ND | 0 | ND |
| 3S | 0 | ND | 0 | ND | 0 | ND |
| 4S | 0 | ND | 0 | ND | 1 | 0/1 |
| 5S | 0 | ND | 0 | ND | 1 | 1/1 |
| 6S | 0 | ND | 0 | ND | 0 | ND |

SP2/0 MP-hTERT

| | | | | | | |
|---|---|---|---|---|---|---|
| 3T | 0 | ND | 2 | 2/2 | 3 | 3/3 |
| 4T | 7 | 7/7 | 21 | 10/10 | 35 | 30/30 |
| ST-1 | 4 | 0/4 | 7 | 0/7 | 19 | 2/19 |
| 5T-2 | 18 | 0/18 | 38 | 0/38 | 46 | 0/46 |
| 6T-1 | 0 | ND | 0 | ND | 2 | 1/2 |
| 6T-2 | 0 | ND | 2 | 2/2 | 3 | 3/3 |

ND: not done
# positive: cells expressing human immunoglobulins by ELISA

TABLE 2
Colony counts following fusion of the murine SP2/O cell line with
murine B-cells.

| | SP2/0-hTERT | SP2/0-hTERT | SP2/0 | SP2/0 |
|---|---|---|---|---|
| Colony size: | Large | Small | Large | Small |
| F1 | 2 | 4 | 2 | 1 |
| F2 | 2 | 1 | 0 | 2 |
| F3 | 2 | 8 | 0 | 0 |
| F4 | 3 | 1 | 3 | 1 |
| F5 | 1 | 3 | 5 | 3 |
| F6 | 3 | 4 | 1 | 6 |
| F7 | 5 | 7 | 1 | 5 |
| F8 | 2 | 2 | 0 | 3 |
| A1 | 6 | 1 | 1 | 1 |
| A2 | 3 | 4 | 0 | 2 |
| A3 | 1 | 3 | 0 | 1 |
| A4 | 2 | 4 | 0 | 0 |
| A5 | 4 | 3 | 0 | 3 |
| A6 | 5 | 1 | 0 | 2 |
| A7 | 2 | 4 | 1 | 0 |
| A8 | 0 | 3 | 1 | 3 |
| Totals | 43 | 52 | 15 | 33 |

### REFERENCES

1. B. Alberts, et al., in Molecular Biology of the Cell. (Garland Publishing, Inc., 1994).

2. E. Harlow, D. Lane, Antibodies: A laboratory manual. (Cold Spring Harbor Laboratory, Cold Spring Harbor, 1988).

3. G. Kohler, C. Milstein. Continuous cultures of fused cells secreting antibody of predefined specificity. Nature 256, 495-7 (1975).

4. N. Chiorazzi, R. L. Wasserman, H. G. Kunkel. Use of Epstein-Barr virus-transformed B cell lines for the generation of immunoglobulin-producing human B cell hybridomas. J Exp Med 156, 930-5 (1982).

5. C. M. Croce, A. Linnenbach, W. Hall, Z. Steplewski, H. Koprowski.

Production of human hybridomas secreting antibodies to measles virus. Nature 288, 488-9 (1980).

6. P. A. Edwards, C. M. Smith, A. M. Neville, M. J. O'Hare. A human-hybridoma system based on a fast-growing mutant of the ARH-77 plasma cell leukemia-derived line. Eur J Immunol 12, 641-8 (1982).

7. R. Nowinski, et al. Human monoclonal antibody against Forssman antigen. Science 210, 537-9 (1980).

8. L. Olsson, H. S. Kaplan. Human-human hybridomas producing monoclonal antibodies of predefined antigenic specificity. Proc Natl Acad Sci USA 77, 5429-31 (1980).

9. J. W. Pickering, F. B. Gelder. A human myeloma cell line that does not express immunoglobulin but yields a high frequency of antibody-secreting hybridomas. J Immunol 129, 406-12 (1982).

10. N. P. Weng, K. S. Hathcock, R. J. Hodes. Regulation of telomere length and telomerase in T and B cells: a mechanism for maintaining replicative potential. Immunity 9, 151-7 (1998).

11. C. K. Borrebaeck, A. C. Malmborg, M. Ohlin. Does endogenous glycosylation prevent the use of mouse monoclonal antibodies as cancer therapeutics? Immunol Today 14, 477-9 (1993).

12. R. A. Greenberg, R. C. Allsopp, L. Chin, G. B. Morin, R. A. DePinho. Expression of mouse telomerase reverse transcriptase during development, differentiation and proliferation. Oncogene 16, 1723-30 (1998).

13. A. G. Bodnar, et al. Extension of life-span by introduction of telomerase into normal human cells. Science 279, 349-52 (1998).

14. T. de Lange. Telomeres and senescence: ending the debate. Science 279, 334-5 (1998).

15. Y. Ishii, N. Tsuyama, S. Maeda, H. Tahara, T. Ide. Telomerase activity in hybrids between telomerase-negative and telomerase-positive immortal human cells is repressed in the different complementation groups but not in the same complementation group of immortality. Mech Ageing Dev 110, 175-93 (1999).

16. M. Katoh, M. Kameyama, H. Kugoh, M. Shimizu, M. Oshimura. A repressor function for telomerase activity in telomerase-negative immortal cells. Mol Carcinog 21, 17-25 (1998).

17. W. E. Wright, D. Brasiskyte, M. A. Piatyszek, J. W. Shay. Experimental elongation of telomeres extends the lifespan of immortal x normal cell hybrids. Embo J 15, 1734-41 (1996).

18. M. Shulman, C. D. Wilde, G. Kohler. A better cell line for making hybridomas secreting specific antibodies. Nature 276, 269-70 (1978).

19. R. K. Naviaux, E. Costanzi, M. Haas, I. M. Verma. The pCL vector system: rapid production of helper-free, high-titer, recombinant retroviruses. J Virol 70, 5701-5 (1996).

20. T. M. Nakamura, et al. Telomerase catalytic subunit homologs from fission yeast and human. Science 277, 955-9 (1997).

21. N. W. Kim, F. Wu. Advances in quantification and characterization of telomerase activity by the telomeric repeat amplification protocol (TRAP). Nucleic Acids Res 25, 2595-7 (1997).

While this invention has been particularly shown and described with references to preferred embodiments thereof, it will be understood by those skilled in the art that various changes in form and details may be made therein without departing from the spirit and scope of the invention as defined by the appended claims. Those skilled in the art will recognize or be able to ascertain using no more than routine experimentation, many equivalents to the specific embodiments of the invention described specifically herein. Such equivalents are intended to be encompassed in the scope of the claims.

What is claimed is:

1. A method of producing a hybrid cell that expresses an ectopic telomerase gene comprising fusing a fusion partner cell with a fusion cell under conditions appropriate for the production of a hybrid cell, wherein the ectopic telomerase gene is introduced into one of the cells and is expressed in the hybrid cell, thereby producing a hybrid cell that expresses an ectopic telomerase gene.

2. A method of claim 1 wherein the ectopic telomerase gene is introduced into the fusion partner cell prior to fusing the fusion partner cell with a fusion cell.

3. A method of claim 1 wherein the ectopic telomerase gene is introduced into the fusion cell prior to fusing the fusion cell with a fusion partner cell.

4. A method of claim 1 wherein the ectopic telomerase gene is introduced into the fusion partner cell, fusion cell, or hybrid cell during the cell fusion.

5. A method of claim 1 wherein the ectopic telomerase gene is introduced into the hybrid cell.

6. The method of claim 1, wherein the fusion partner cell is an immortal mammalian cell.

7. The method of claim 6, wherein the fusion partner cell is a human cell or a murine cell.

8. The method of claim 6, wherein the fusion partner cell is a B-lineage cell.

9. The method of claim 6, wherein the fusion partner cell is a myeloma cell line.

10. The method of claim 1, wherein the fusion cell is a B-lineage cell.

11. The method of claim 10, wherein the B-lineage cell is of human origin.

12. The method of claim 1, wherein the telomerase gene is the human telomerase gene.

13. A hybrid cell produced by the method of claim 1.

14. A hybrid cell of claim 13 that ectopically expresses human telomerase.

15. A hybrid cell of claim 13 that ectopically expresses telomerase and produces antibody molecules.

16. A hybrid cell of claim 13, wherein the antibody molecules produced may include immunoglobulin A, immunoglobulin E, immunoglobulin G, immunoglobulin M, or portions and derivatives thereof.

17. A hybrid cell of claim 16 that produces human antibody molecules.

18. A hybrid cell of claim 16 that produces antibody molecules encoded in full or in part by genes originating from the human B-cell fusion cell.

19. A hybrid cell produced by fusing: (a) a mammalian fusion partner that ectopically expresses telomerase and (b) a human B-lymphocyte, wherein the hybrid cell expresses an antibody derived from the human B-lymphocyte.

20. An antibody produced by the hybrid cell of claim 19.

21. A method of producing a hybrid cell that expresses an ectopic telomerase gene comprising fusing an immortal mammalian cell line that expresses an ectopic telomerase gene with a fusion cell under conditions appropriate for the production of hybrid cells, thereby producing a hybrid cell that expresses an ectopic telomerase gene.

22. The method of claim 21, wherein the fusion partner cell is an immortal mammalian cell.

23. The method of claim 22, wherein the fusion partner cell is a human cell or a murne cell.

24. The method of claim 22, wherein the fusion partner cell is a B-lineage cell.

25. The method of claim 22, wherein the fusion partner cell is a myeloma cell line.

26. The method of claim 21, wherein the fusion cell is a B-lineage cell.

27. The method of claim 26, wherein the B-lineage cell is of human origin.

28. The method of claim 21, wherein the telomerase gene is the human telomerase gene.

29. A hybrid cell produced by the method of claim 21.

30. A hybrid cell of claim 29 that ectopically expresses human telomerase.

31. A hybrid cell of claim 29 that ectopically expresses telomerase and produces antibody molecules.

32. A hybrid cell of claim 29, wherein the antibody molecules produced may include immunoglobulin A, immunoglobulin E, immunoglobulin G, immunoglobulin M, or portions and derivatives thereof.

33. A hybrid cell of claim 32 that produces human antibody molecules.

34. A hybrid cell of claim 16 that produces antibody molecules encoded in full or in part by genes originating from the human B-cell fusion cell.

35. An immortal mammalian cell line that expresses an endogenous telomerase gene and an ectopic telomerase gene.

36. The immortal mammalian cell line of claim 35, wherein the cell line is of human or murine origin.

37. The immortal mammalian cell line of claim 36 which has been modified to ectopically express telomerase constitutively by expressing a telomerase gene that is expressed from a constitutively active promoter.

38. An immortal mammalian cell line of B-lineage that expresses an endogenous telomerase gene and an ectopic telomerase gene.

39. The immortal mammalian cell line of claim 38, wherein the cell line is of human or murine origin.

40. The immortal mammalian cell line of claim 39, wherein the ectopically expressed telomerase gene is constitutively expressed.

41. The immortal mammalian cell line of claim 40, wherein the constitutively active promoter is selected from the group consisting of: a viral promoter, a eukaryotic promoter, a prokaryotic promoter and a synthetic promoter.

42. The immortal mammalian cell line of claim 38, wherein the cells of B cell lineage express Epstein-Barr Virus antigens.

43. An immortal mammalian B-cell, for production of hybridomas, wherein telomerase is expressed ectopically.

44. The immortal mammalian B-cell of claim 43 that is a murine immortal B-cell or a human immortal B-cell.

45. The immortal mammalian B cell of claim 44 that has been modified to ectopically express telomerase constitutively by expressing a telomerase gene that is expressed from a constitutively active promoter.

46. An immortal mammalian lymphoblastoid cell that ectopically expresses telomerase.

47. A method of producing a hybridoma that ectopically expresses telomerase, comprising fusing an immortal mammalian cell line that ectopically expresses telomerase with a fusion partner, under conditions appropriate for production of hybridomas, thereby producing a hybridoma that ectopically expresses telomerase.

48. The method of claim 47, wherein the immortal mammalian cell line is a human cell line or a murine cell line and the fusion cell is a human cell.

49. The method of claim 48, wherein the immortal mammalian cell line is a myeloma cell line and the fusion partner is a B-lineage cell.

50. The method of claim 27, wherein the myeloma cell line is a murine cell line and the B-lineage cell is an antigen-stimulated human peripheral blood mononuclear cell.

51. A hybridoma produced by the method of claim 47.

52. A hybridoma produced by the method of claim 48.

53. A hybridoma produced by the method of claim 49.

54. A hybridoma produced by the method of claim 50.

55. A method of producing human monoclonal antibodies, comprising maintaining a hybidoma that ectopically expresses telomerase under conditions appropriate for the production of monoclonal antibodies by the hybridoma, thereby producing monoclonal antibodies.

56. A method of producing human monoclonal antibodies, comprising: (a) fusing an immortal mammalian cell line that ectopically expresses telomerase with a human fusion partner, under conditions appropriate for hybridoma formation, thereby producing hybridomas that ectopically express telomerase and (b) maintaining hybridomas produced in (a) under conditions appropriate for production of monoclonal antibodies by the hybridomas, whereby human monoclonal antibodies are produced.

57. The method of claim 56, wherein the immortal mammalian cell line has been modified to ectopically express telomerase constitutively by expressing a telomerase gene that is expressed from a constitutively active promoter.

58. The method of claim 57, wherein the immortal mammalian cell line is of human or murine origin.

59. A hybridoma that ectopically expresses telomerase.

60. The hybridoma of claim 59 which is a murine hybridoma, a human hybridoma, or a murine/human hybridoma.

61. A method of producing a hybridoma that produces antibodies that bind an antigen expressed by a malignant cell, comprising fusing an immortal cell line that ectopically expresses telomerase with a fusion cell that is a malignant cell, under conditions appropriate for formation of hybridomas, whereby a hybridoma that produces antibodies that bind the antigen expressed by the malignant cell is produced.

62. The method of claim 61, wherein the malignant cell is selected from the group consisting of: a solid malignant tumor cell and a hematopoietic tumor cell.

63. The method of claim 62, wherein the solid tumor is selected from the group consisting of: a gastrointestinal tumor, a breast tumor, a kidney tumor, a brain tumor, a liver tumor, a stomach tumor, a lung tumor, a pancreatic tumor, a tumor of the reproductive systems, a prostate tumor, an eye tumor, a skin tumor, a melanoma, adenomas, polyps, dysplasias, in situ carcinomas, and intra-epithelial neoplasms and the hematopoietic tumor cell is selected from the group consisting of: leukemia, lymphoma, or myeloma, and myelodysplastic syndromes.

64. A method of producing a hybridoma that expresses antibodies that bind an antigen expressed by a pathogen, comprising fusing an immortal cell line that ectopically expresses telomerase with fusion cells that are B-lineage cells from an individual who is or has been infected with the pathogen, under conditions appropriate for formation of hybridomas, whereby a hybridoma that produces antibodies that bind the antigen expressed by the pathogen is produced.

65. The method of claim 45, wherein the pathogen is selected from the group consisting of: a RNA virus, a DNA virus, a bacterium, an intracellular parasite, a fungus, a helminth and a protozoan.

66. The method of claim 65, wherein the RNA virus is a member of a RNA virus family selected from the group consisting of: Picomaviridae, Calciviridae, Togaviridae, Flaviviridae, Coronaviridae, Rhabdoviridae, Filoviridae, Paramyxoviridae, Orthomyxoviridae, Bunyaviridae, Arenaviridae, Reoviridae and Retroviridae

67. The method of claim 65, wherein the DNA virus is a member of a DNA virus family selected from the group consisting of: Hepadnaviridae, Parvoviridae, Papovaviridae, Adenoviridae, Herpesviridae and Poxviridae and Hepatitis.

68. The method of claim 65, wherein the bacterium is selected from the group consisting of: gram-positive cocci, gram positive bacilli, gram-negative bacteria, anaerobic bacteria, organisms of the families Actinomycetaceae, Bacillaceae, Bartonellaceae, Bordetellae, Captophagaceae, Corynebacteriaceae, Enterobacteriaceae, Legionellaceae, Micrococcaceae, Mycobacteriaceae, Nocardiaceae, Pasteurellaceae, Pseudomonadaceae, Spirochaetaceae, Vibrionaceae and organisms of the genera Acinetobacter, Brucella, Campylobacter, Erysipelothrix, Ewingella, Francisella, Gardnerella, Helicobacter, Levinea, Listeria, Streptobacillus and Tropheryma.

69. The method of claim 65, wherein the intracellular parasite is selected from the group consisting of: Chlamydiaceae, Mycoplasmataceae, Acholeplasmataceae, Rickettsiae and organisms of the genera Coxiella and Ehrlichia.

70. The method of claim 65, wherein the fungus is selected from the group consisting of: Aspergillus, Blastomyces, Candida, Coccidioides, Cryptococcus, Histoplasma, Paracoccicioides, Sporothrix, organisms of the order Mucorales, organisms inducing choromycosis and mycetoma and organisms of the genera Trichophyton, Microsporum, Epidermophyton, and Malassezia.

71. The method of claim 65, wherein the helminth is selected from the group consisting of: Nematodes, Trematodes and Cestodes.

72. The method of claim 65, wherein the protozoan is selected from the group consisting of: organisms of the phylum Sarocomastigophora, the phylum Apicomplexa, the phylum Ciliophora, the phylum Microspora, and Pneumocystis carinii.

73. A method of producing a hybridoma that produces antibodies that bind an antigen expressed by a malignant cell, comprising fusing an immortal cell line that ectopically expresses telomerase with a fusion cell that is a malignant cell, under conditions appropriate for formation of hybridomas, whereby a hybridoma that produces antibodies that bind the antigen expressed by the malignant cell is produced.

74. A method of producing a hybridoma that produces antibodies that bind a self-antigen, comprising fusing an immortal cell line that ectopically expresses telomerase with a fusion cell obtained from a person who is or has been affected by an autoimmune disease, under conditions appropriate for formation of hybridomas, whereby a hybridoma that produces antibodies that bind the self-antigen is produced.

75. A method of producing a hybridoma that produces antibodies that bind a prion antigen, comprising fusing an immortal cell line that ectopically expresses telomerase with a fusion cell obtained from a person who is or has been affected by a prion disease, under conditions appropriate for formation of hybridomas, whereby a hybridoma that produces antibodies that bind the prion antigen is produced.

76. A method of producing a hybridoma that produces antibodies that bind an antigen in an antigen preparation, comprising fusing an immortal cell line that ectopically expresses telomerase with a fusion cell that has been stimulated in vitro in the presence the antigen preparation, under conditions appropriate for formation of hybridomas, whereby a hybridoma that produces antibodies that bind an antigen in the antigen preparation is produced.

77. A DNA construct useful for introducing DNA that encodes telomerase into a mammalian cell to modify the cell to ectopically express telomerase, comprising: (a) a telomerase gene; and (b) DNA that undergoes homologous recombination with a region of genomic DNA of the mammalian cell in such a manner that introduction of the telomerase gene into the genomic DNA of the mammalian cell places it under control of transcription regulatory elements of the mammalian cell that direct constitutive expression of the telomerase gene in the mammalian cell.

78. The DNA construct of claim 77 which is a plasmid or a viral vector.

79. A DNA construct useful for introducing DNA to modify a mammalian cell to ectopically express an endogenous telomerase gene, comprising: a constitutively active promoter flanked by DNA that undergoes homologous recombination with the genomic DNA of the mammalian cell in such a manner that the constitutively active promoter is introduced into a site from which it directs the constitutive ectopic expression of an endogenous telomerase gene in the mammalian cell.

80. The DNA construct of claim 79, wherein the DNA that undergoes homologous recombination with genomic DNA is homologous to DNA of the endogenous telomerase gene promoter.

81. The DNA construct of claim 80 which is a plasmid or a viral vector.


SEQUENCE LISTING (6 sequences)

SEQ ID NO: 1 - DNA, 18 bp, Artificial Sequence
  AN1 forward primer
tgagcggacaaaacatcc

SEQ ID NO: 2 - DNA, 20 bp, Artificial Sequence
  AC1 reverse primer
aggctcgtcttaattgaggt

SEQ ID NO: 3 - DNA, 20 bp, Artificial Sequence
  LT5 forward primer
cggaagagtgtctggagcaa

SEQ ID NO: 4 - DNA, 19 bp, Artificial Sequence
  LT6 reverse primer
ggatgaagcggagtctgga

SEQ ID NO: 5 - DNA, 21 bp, Artificial Sequence
  GAPDH1 forward primer
gaccccttcattgacctcaac

SEQ ID NO: 6 - DNA, 20 bp, Artificial Sequence
  GAPDH2 reverse primer
cttctccatggtggtgaaga